(19) United States
(12) Patent Application Publication
(10) Pub. No.: US 2023/0181569 A1
(43) Pub. Date: Jun. 15, 2023

(54) COMPOSITIONS AND METHODS TO TREAT POLYCYSTIC KIDNEY DISEASE

(71) Applicant: Board of Supervisors of Louisiana State University and Agricultural and Mechanical College, Baton Rouge, LA (US)

(72) Inventors: Juan Gao, New Orleans, LA (US); Daniel Kapusta, Slidell, LA (US); Mihran Naljayan, New Orleans, LA (US); Jacob K. Meariman, River Ridge, LA (US)

(21) Appl. No.: 17/922,727

(22) PCT Filed: May 3, 2021
(86) PCT No.: PCT/US2021/030445

§ 371 Date: Nov. 1, 2022

Related U.S. Application Data
(60) Provisional application No. 63/018,994, filed on May 1, 2020.

(51) Int. Cl. A61K 31/485; A61P 13/12; A61K 38/07; A61K 38/06

(52) U.S. Cl. CPC A61K 31/485; A61K 38/06; A61K 38/07; A61P 13/12

(57) ABSTRACT

This invention is directed to compositions and methods of treating polycystic kidney disease by administering a kappa opioid receptor agonist (KOA). For examples, this invention is directed to compositions and methods of treating polycystic kidney disease by reducing circulating blood levels of vasopressin

This application claims priority from U.S. Provisional Application No. 63/018,994 filed on May 01, 2020, the entire contents of which is hereby incorporated by referenced.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### FIELD OF THE INVENTION

This invention is directed to compositions and methods of treating polycystic kidney disease by administering kappa opioid receptor agonists (KOA). For examples, this invention is directed to compositions and methods of treating polycystic kidney disease by reducing circulating blood levels of vasopressin.

### BACKGROUND OF THE INVENTION

Polycystic kidney disease (PKD) is a pathology characterized with variable size cysts growing in the kidneys, which replace normal nephron tissue, structure, and function. Water and electrolyte imbalance, including fluid overload and hyponatremia, can occur in patients with PKD. Progression of the disease leads to chronic renal failure and need for dialysis and kidney transplantation.

### SUMMARY OF THE INVENTION

The invention provides a method of treating polycystic kidney disease (PKD). For example, the polycystic kidney disease comprises autosomal dominant polycystic kidney disease (ADPKD). In another example, the polycystic kidney disease is autosomal recessive polycystic kidney disease.

The invention further provides a method of decreasing circulating levels of vasopressin. For example, decreasing circulating levels of vasopressin can treat polycystic kidney disease.

In embodiments, the method comprises administering to a subject a composition that decreases circulating levels of vasopressin. For example, the composition comprises a kappa opioid receptor agonist (KOA). For example, the composition comprises a nociceptin opioid peptide (NOP) receptor agonist. The composition can be administered centrally. The composition can be administered peripherally. For example, the kappa opioid receptor agonist is administered centrally. In another example, the kappa opioid receptor agonist is administered peripherally. For example, the NOP receptor agonist is administered centrally. In another example, the NOP receptor agonist is administered peripherally. For example, the peripherally administered NOP receptor agonist or peripherally administered kappa opioid receptor agonists can cross the blood brain barrier.

In embodiments, the composition can be administered by bolus injection, such as bolus intraneous injection, intramuscular injection, nasal injection (such as intranasal spray), infusion, such as by intravenous infusion, or by oral administration.

In embodiments, the composition can be administered chronically.

In embodiments, the composition can be administered as a pharmaceutical composition.

Referring to the kappa opioid receptor agonist, any pharmacologically acceptable kappa opioid agonist will function in the invention. Such kappa opioid receptor agonists bind to and stimulate kappa opioid receptors.

In one embodiment, the kappa opioid receptor agonist comprises an organic small molecule. Non-limiting examples of organic small molecules comprise JT09, nalfurafine, CR665, or CR845.

In another embodiment, a therapeutically effective amount of a kappa opioid receptor agonist (KOA) is between about 0.005 mg/kg and about 10 mg/kg.

In one embodiment, the organic small molecule comprises the molecule of:

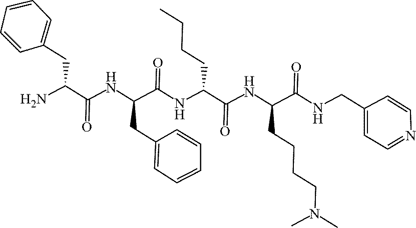 [SMILES: CCCC[C@@H](NC(=O)[C@@H](Cc1ccccc1)NC(=O)[C@H](N)Cc1ccccc1)C(=O)N[C@H](CCCCN(C)C)C(=O)NCc1ccncc1]

In another embodiment, the organic small molecule comprises a molecule of:

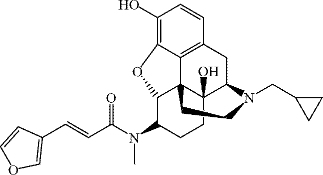

In another embodiment, the organic small molecule comprises a molecule of:

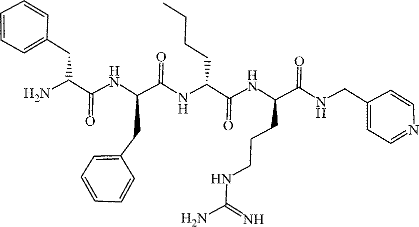

In another embodiment, the organic small molecule comprises a molecule of:

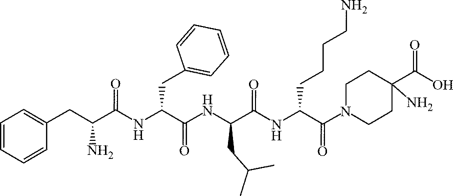 [SMILES: CC(C)C[C@@H](NC(=O)[C@@H](Cc1ccccc1)NC(=O)[C@H](N)Cc1ccccc1)C(=O)N[C@H](CCCCN)C(=O)N1CCC(N)(C(=O)O)CC1]

Referring to the Nociceptin Opioid Peptide Receptor (NOP), any pharmaceutically acceptable agonist will function in the invention. Such NOP receptor agonists bind to and stimulate NOP receptors.

Non-limiting examples of NOP agonists comprises nociceptin and peptide-conjugates thereof.

Other NOP agonists comprise a chemical structure according to Structure (V):

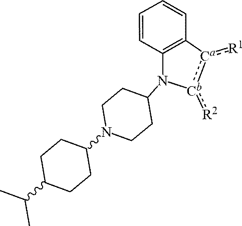

wherein

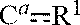

is a single bond and R¹ is CH₂—CH═O—NH—CH₃, CH₂—OH, alkyl, OH, or H; and

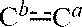

is a double bond and

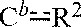

is a single bond and R² is CH₂—OH, OH, H, or alkyl; or

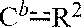

is a single bond and

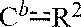

is a double bond and R² is O; or

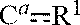

is a double bond and R¹ is O, N—OH; and

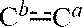

is a single bond; and

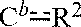

is a double bond and R² is O.

For example, non-limiting examples of NOP agonists comprises AT-390, AT-090, AT-127, and AT-403.

For example, the NOP agonist can comprise a structure according to Structure (VI):

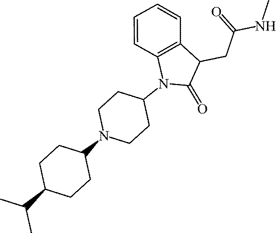

For example, the NOP agonist can comprise a structure according to Structure (VII):

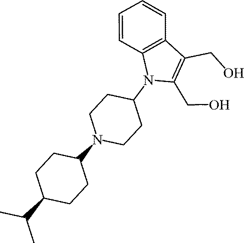

For example, the NOP agonist can comprise a structure according to Structure (XI):

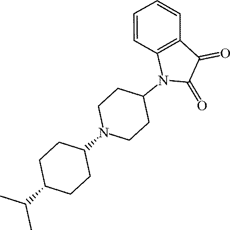

For example, the NOP agonist can comprise a structure according to Structure (VIII):

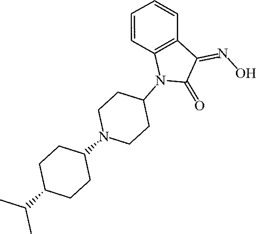

In one embodiment, treating, such as treating polycystic kidney disease, comprises decreasing circulating blood levels of vasopressin, maintaining glomerular filtration rate, reducing kidney fibrosis, decreasing blood urea levels, decreasing cysts number, decreasing cysts size, decreasing serum creatinine levels, or a combination thereof.

Other objects and advantages of this invention will become readily apparent from the ensuing description.

### BRIEF DESCRIPTION OF THE FIGURES

FIG. 1 shows acute study of oral gavage of Nalfurafine.

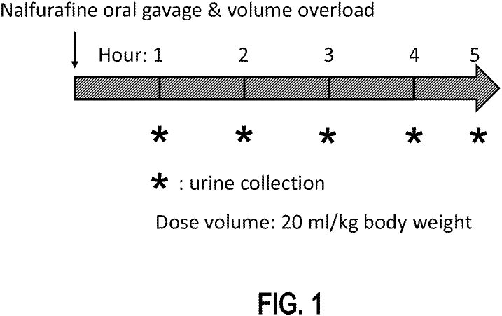

FIG. 2 shows oral gavage of Nalfurafine in >1 yr old WT mice. The dose volume is 20 ml/kg.

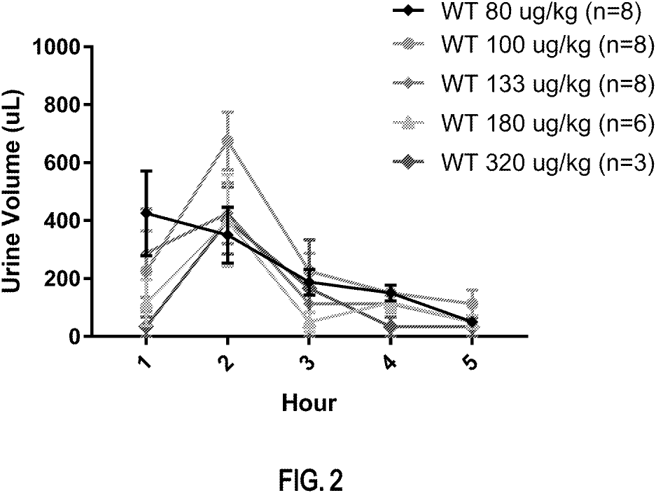

FIG. 3 shows oral gavage of Nalfurafine in 4 months old PKD mice.

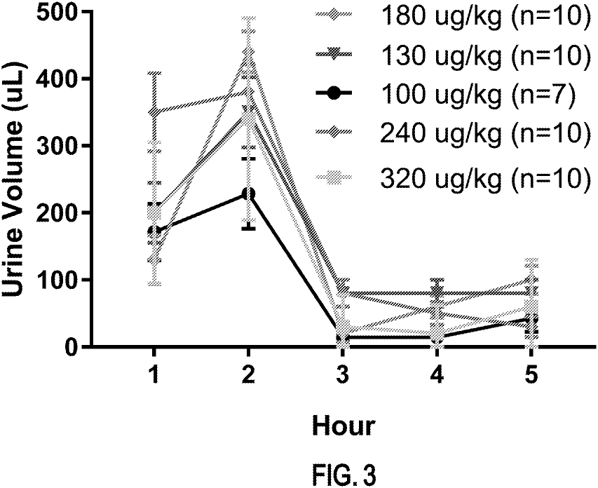

FIG. 4 shows oral gavage of Nalfurafine in 4 months old PKD mice.

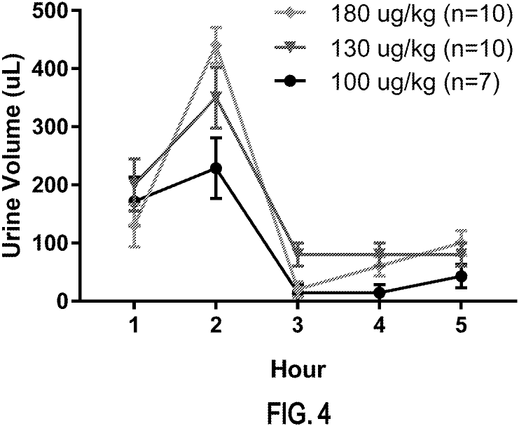

FIG. 5 shows dose response curve versus accumulated urine output in the total 5 hours in acute study of PKD mice.

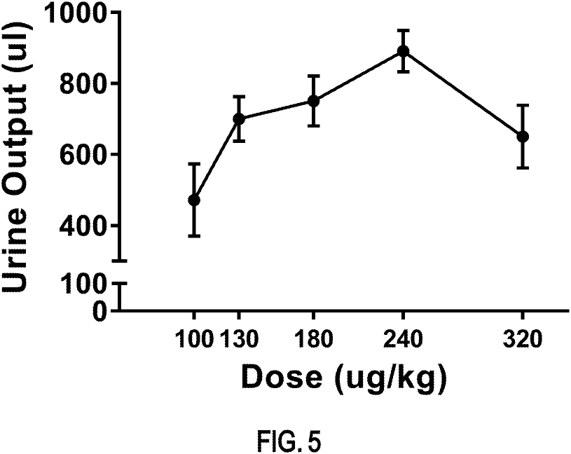

FIG. 6 shows daily oral gavage of nalfurafine at 0, 100 and 180 ug/kg for 2 weeks in 5 months old PKD mice.

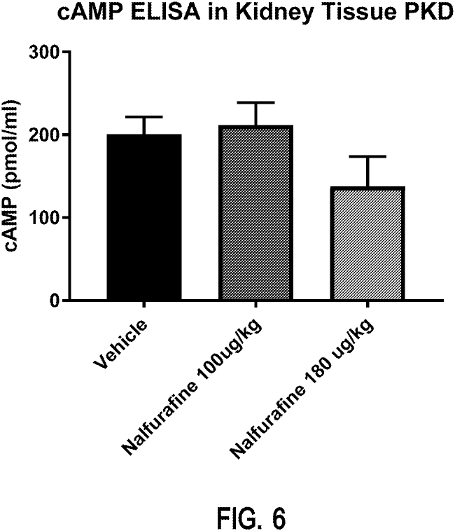

FIG. 7 shows chronic administration of nalfurafine via miniosmotic pumps at 60 ug/kg/hr for 1 month on 1-year-old PKD mice. GFR: measure FITC-sinistrin clearance.

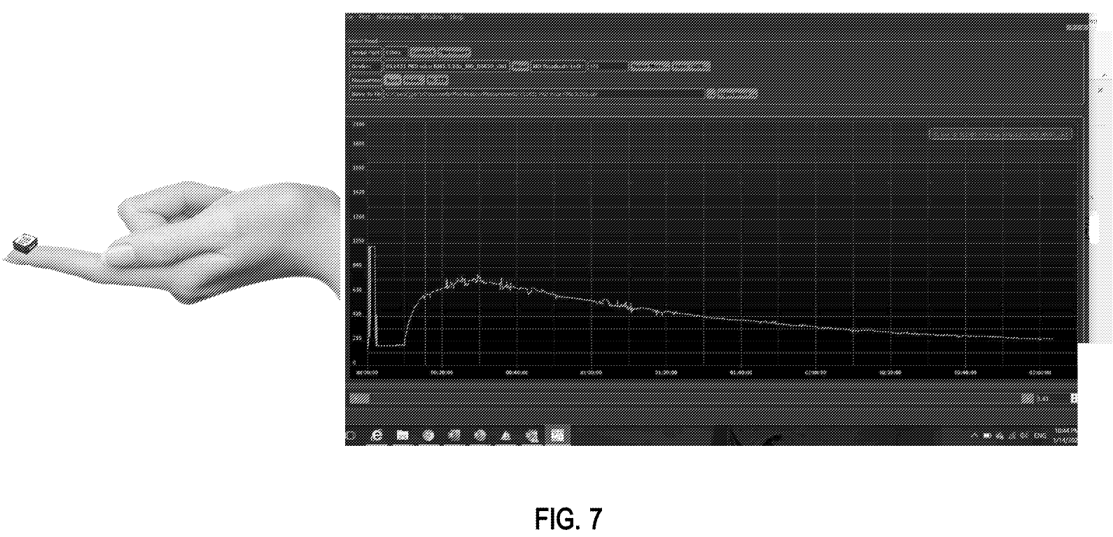

FIG. 8 shows chronic administration of nalfurafine via miniosmotic pumps at 60 ug/kg/hr for 1 month on 1-year-old PKD mice. The dose of nalfurafine was decided by the half life of nalfufafine, which in healthy volunteers is 8 hours, and 10-15 hours in hemodialysis patients. The highest effective does in acute study is 240 ug/kg. n=1, nalfurafine; n=4, vehicle.

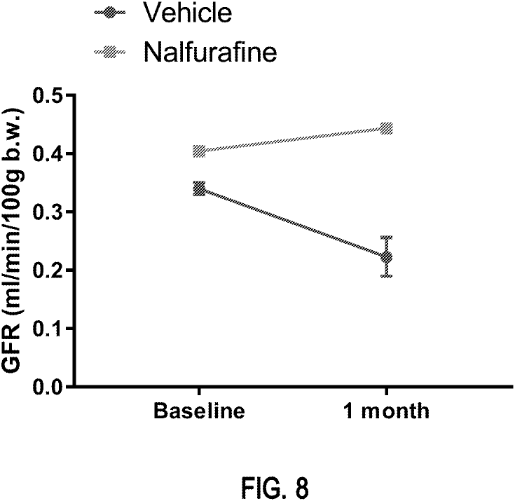

FIG. 9 shows chronic administration of nalfurafine via miniosmotic pumps at 60 ug/kg/hr for 1 month on 1-year-old PKD mice. Cyclic-AMP (cAMP) levels in kidney tissue.

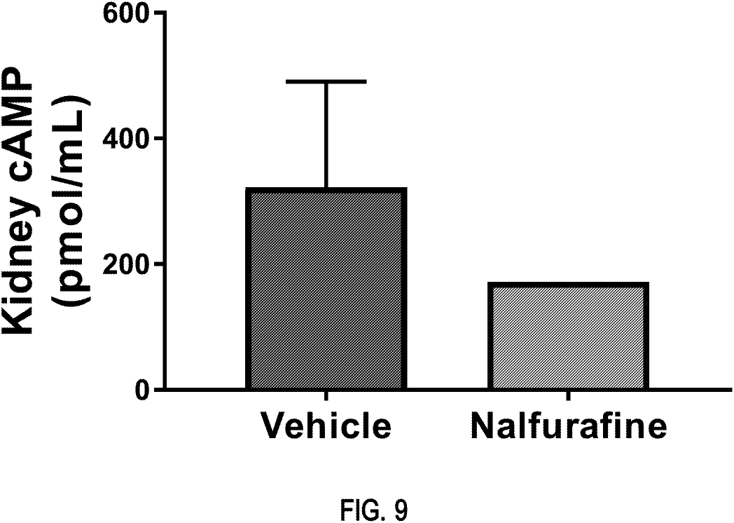

FIG. 10 shows chronic administration of nalfurafine via miniosmotic pumps at 60 ug/kg/hr for 2 months on 9-month-old PKD mice. N=3 for each group. GFR.

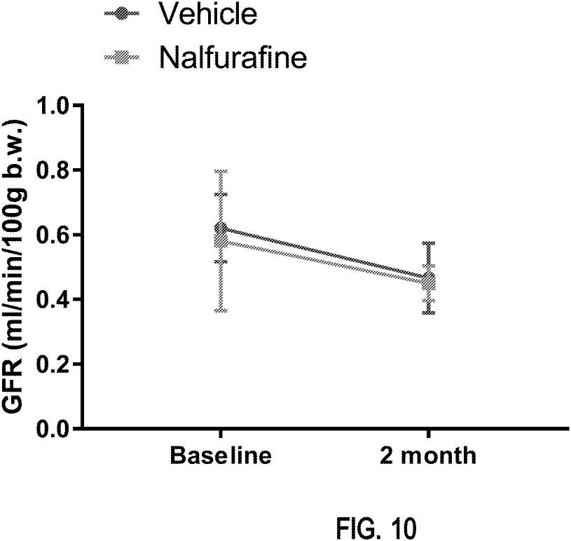

FIG. 11 shows chronic administration of nalfurafine via miniosmotic pumps at 60 ug/kg/hr for 2 months on 9-month-old PKD mice. N=3 for each group. Total kidney volume and kidney weight.

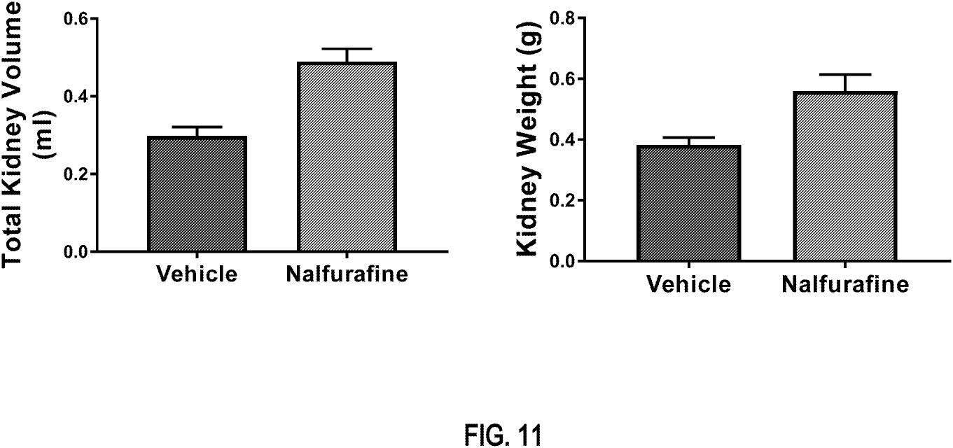

FIG. 12 shows chronic administration of nalfurafine via miniosmotic pumps at 60 ug/kg/hr for 2 months on 9-month-old PKD mice. 24 hour metabolic cage study to get urine output and water intake.

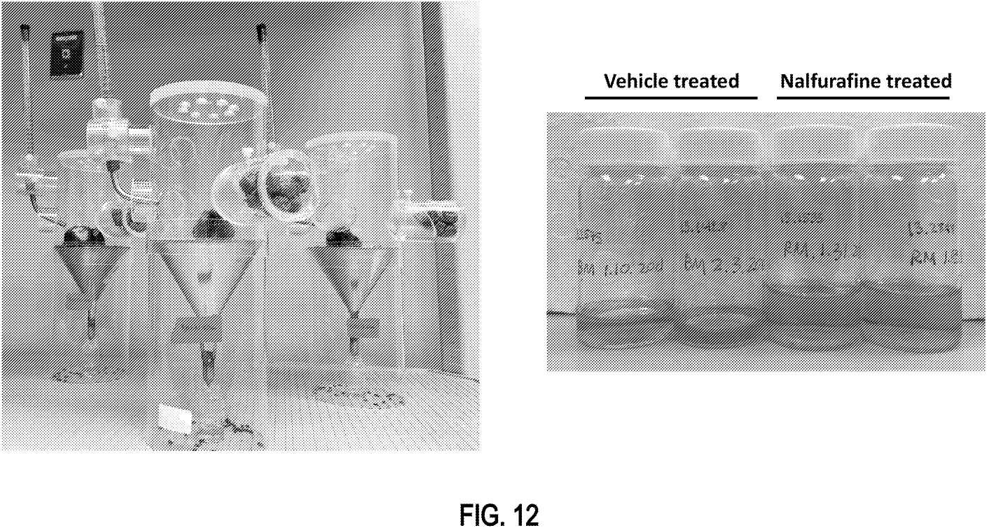

FIG. 13 shows chronic administration of nalfurafine via miniosmotic pumps at 60 ug/kg/hr for 2 months on 9-month-old PKD mice. N=3 for each group. Free water clearance, plasma and urine osmolarity, and urine volume within 24 hours.

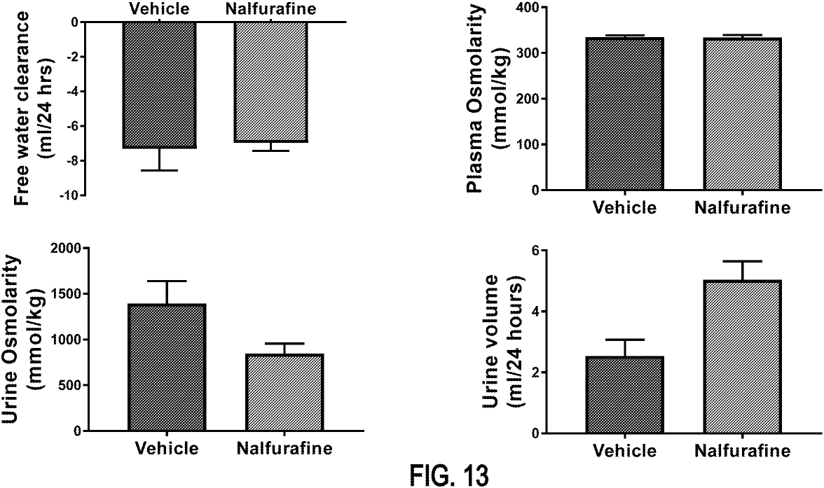

FIG. 14 shows chronic administration of nalfurafine via miniosmotic pumps at 60 ug/kg/hr for 2 months on 9-month-old PKD mice. N=3 for each group. Urinary sodium and potassium levels.

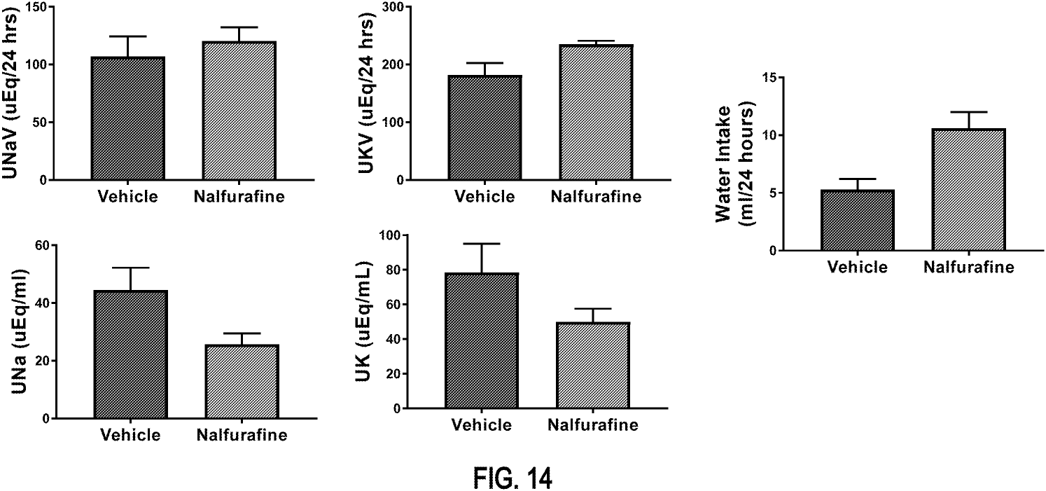

FIG. 15 shows chronic administration of nalfurafine via miniosmotic pumps at 60 ug/kg/hr for 2 months on 9-month-old PKD mice. Cyclic AMP levels in kidney tissue, plasma, and urine.

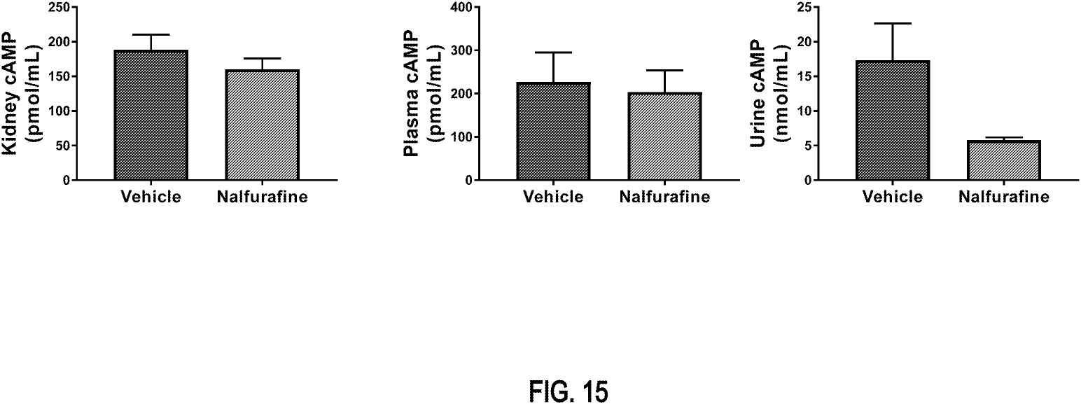

FIG. 16 shows chronic administration of nalfurafine via miniosmotic pumps at 60 ug/kg/hr for 2 months on 9-month-old PKD mice. HE sstaining of kidney sections.

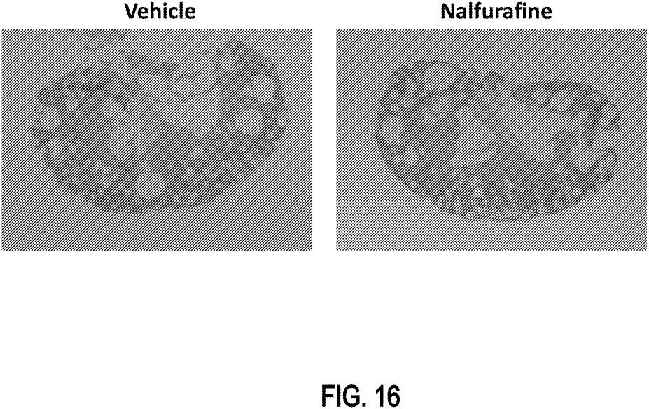

FIG. 17 shows chronic administration of nalfurafine via miniosmotic pumps at 60 ug/kg/hr for 2 months on 6-month-old PKD mice. N=2 for each group. GFR.

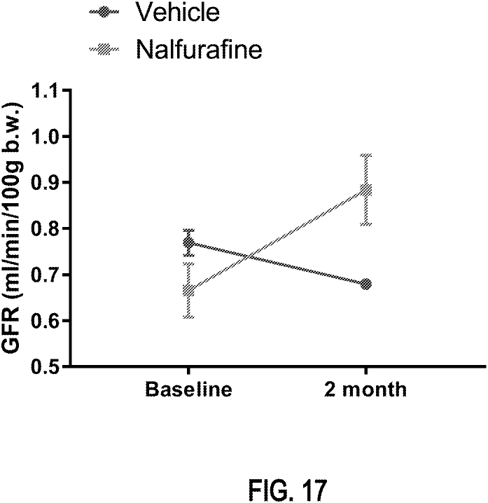

FIG. 18 shows chronic administration of nalfurafine via miniosmotic pumps at 60 ug/kg/hr for 2 months on 6-month-old PKD mice. N=2 for each group. Total kidney volume and kidney weight.

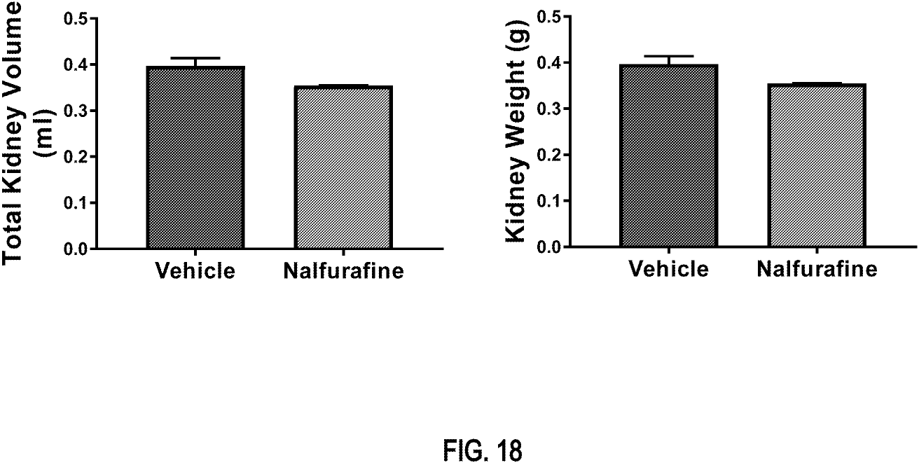

FIG. 19 shows chronic administration of nalfurafine via miniosmotic pumps at 60 ug/kg/hr for 2 months on 6-month-old PKD mice. N=2 for each group. Free water clearance, plasma volume, urine and plasma osmolarity.

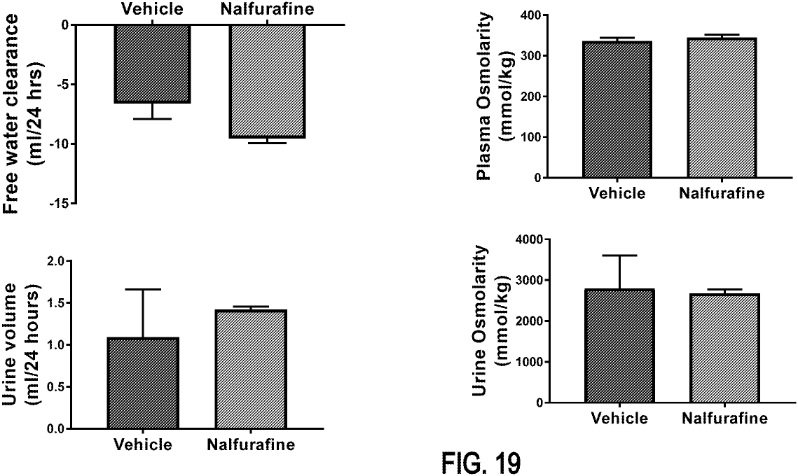

FIG. 20 shows chronic administration of nalfurafine via miniosmotic pumps at 60 ug/kg/hr for 2 months on 6-month-old PKD mice. N=2 for each group. Urinary sodium and potassium levels and water intake within 24 hours.

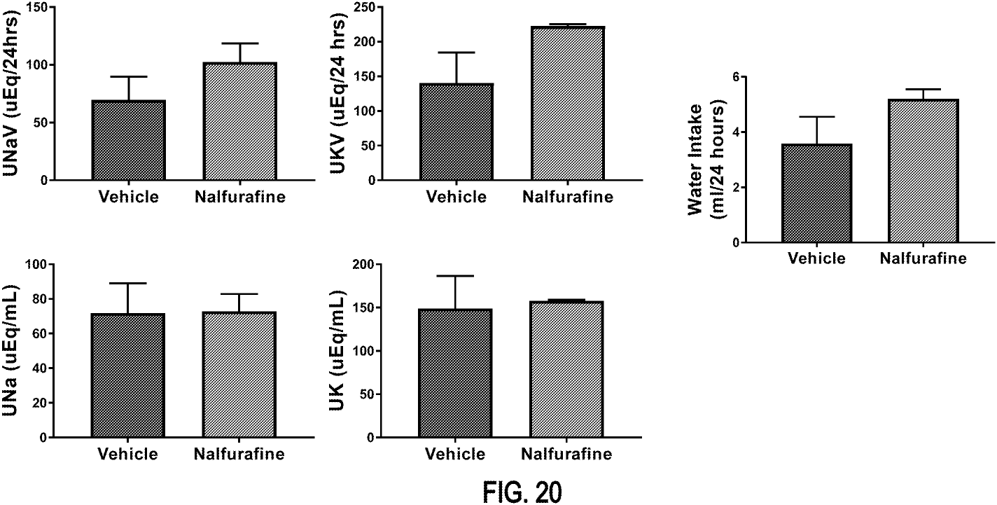

FIG. 21 shows chronic administration of nalfurafine via miniosmotic pumps at 60 ug/kg/hr for 2 months on 6-month-old PKD mice. Cyclic AMP levels in kidney tissue, plasma and urine.

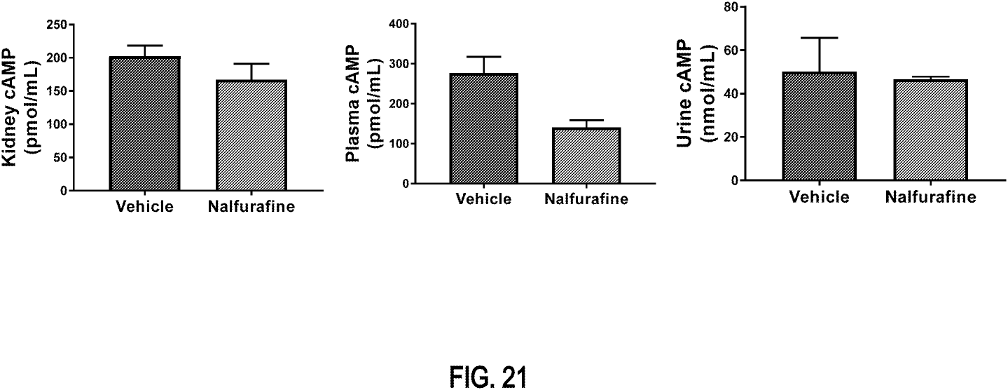

FIG. 22 shows chronic administration of nalfurafine via miniosmotic pumps at 60 ug/kg/hr for 2 months on 6-month-old PKD mice. H&E staining of kidney sections.

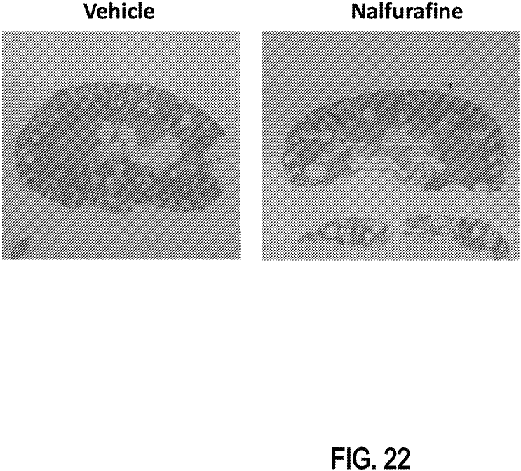

FIG. 23 shows schematic of described studies.

FIG. 24 shows (a) urine output produced by oral gavage of nalfurafine at 240 ug/kg or vehicle in Pkd1^{RC/RC} mice. Volume expansion, give mice saline at the dose of 20 ml/kg. (b) dose responsive curve of nalfurafine in Pkd1^{RC/RC} mice. Y axis, accumulative urine output of 5 hours after dosing. n=7-10 in each group.

FIG. 25 shows urine output after a single dose (30 ug/kg) of nalfurafine in Sprague Dawley rats. Day 1, the fresh made drug was given to rats. Day 4, the drug made on day 1 was given to the same rats. Veh, vehicle; Nal, nalfurafine. n=3 for each group.

FIG. 26 shows experiments outlined herein. US – ultrasound; IHC –immunohistochemistry.

FIG. 27 shows experiments outlined herein. US – ultrasound; GFR – glomerular filtration rate.

### DETAILED DESCRIPTION OF THE INVENTION

In polycystic kidney disease (PKD), elevated levels of vasopressin are a major contributor to cyst growth by stimulating cell proliferation and fluid secretion. Aspects of the invention are drawn to compositions and methods that act to decrease circulating blood levels of vasopressin. Thus, the invention is directed to compositions and methods of treating polycystic kidney disease (PKD). For example, one embodiment comprises administering to a subject a therapeutically effective amount of a composition that reduces circulating blood levels of vasopressin. For example, one embodiment comprises administering to a subject a therapeutically effective amount of a kappa opioid receptor agonist (KOA). For example, one embodiment comprises administering to a subject a therapeutically effective amount of a nociceptin opioid peptide receptor (NOP) agonist. In embodiments, the composition can be administered centrally. In embodiments, the composition can be administered peripherally. Peripherally administered agonists can cross the blood brain barrier. In embodiments, the therapeutically effective amount of the composition can be administered orally or intravenously. Treating can comprise, for example, decreasing circulating blood levels of vasopressin, maintaining glomerular filtration rate, reducing kidney fibrosis, decreasing blood urea levels, decreasing cysts number, decreasing cysts size, decreasing creatinine levels, or a combination thereof.

Detailed descriptions of one or more embodiments are provided herein. It is to be understood, however, that the invention can be embodied in various forms. Therefore, specific details disclosed herein are not to be interpreted as limiting, but rather as a basis for the claims and as a representative basis for teaching one skilled in the art to employ the invention in any appropriate manner.

The singular forms “a”, “an” and “the” include plural reference unless the context clearly dictates otherwise. The use of the word “a” or “an” when used in conjunction with the term “comprising” in the claims and/or the specification can mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly, “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

As used herein the term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

Aspects of the invention are directed towards compositions and methods for treating polycystic kidney disease (PKD). The term “polycystic kidney disease” (PKD) can refer to a genetic disorder characterized by the growth of numerous cysts in the kidney. PKD cysts can slowly reduce much of the mass of kidneys reducing kidney function and leading to kidney failure. PKD can be classified as two major inherited forms of PKD which are autosomal dominant PKD and autosomal recessive PKD, while the non-inherited PKD can be referred to as acquired cystic kidney disease. Biomarkers can be determined and/or measured by non-invasive imaging, such as reduction of renal cysts by non-invasive imaging, or genetic testing, such as genetic testing for PKD1 and PKD2 genes.

In embodiments, the term “treating” or “to treat” can refer to clinical intervention in an attempt to alter the natural course of the individual or subject being treated. Non-For example, “treating a disease” can comprise curing a disease, preventing a disease, reducing the incidence of a disease, ameliorating symptoms of a disease, or slow down the progression of the disease.

Endogenous opioid receptors have been identified in both the central nervous system (brain and spinal cord), and in the periphery. These receptors have been classified into three major subtypes: mu, delta, and kappa receptors. The so-called “kappa opioid agonists,” bind to kappa receptors with high selectivity. A compound is considered a kappa opioid agonist if it binds to kappa receptors in a binding assay, or if it demonstrates kappa agonist activity in functional assays.

Administration of a kappa opioid receptor agonist (KOA) to animals or man causes an increase in urine output (diuresis) often associated with a decrease in urinary sodium/potassium excretion; this pattern of changes in water and electrolytes is referred to as a water diuresis (aquaresis). Each of the following patents, which are incorporated herein by reference in their entireties, discloses that kappa opioid agonists have various characteristics including diuretic properties: Horwell et al., U.S. Pat. Nos. 4,663,343, 4,906,655, 4,965,278, 5,019,588, 5,063,242; Clemence et al., U.S. Pat. Nos. 4,888,355, 4,988,727; Zimmerman et al., U.S. Pat. Nos. 4,891,379, 4,992,450, 5,064,834, 5,319,087, 5,422,356; Naylor et al., U.S. Pat. No. 5,116,842; Moura et al., U.S. Pat. Nos. 5,068,244, 5,130,329; and McKnight et al., U.S. Pat. Nos. 5,317,028, and 5,369,105.

In embodiments, a kappa opioid receptor agonist is administered to a subject. The kappa opioid receptor (KOR) is a G protein-coupled receptor that in humans is encoded by the OPRK1 gene. KORs are widely distributed in the brain, spinal cord, and in peripheral tissues. Based on receptor binding studies, three variants of the KOR referred to as K₁, K₂, and K₃ have been characterized.

The term “agonist” can refer to a compound that binds to a receptor, such as the KOR, and elicits a response in the cell. An “agonist” mimics the action of an endogenous ligand, such as a hormone, peptide, or protein, and causes a physiological response similar to that produced by an endogenous ligand.

The term “partial agonist” can refer to a compound that binds to a receptor and induces a partial response in a cell. Partial agonists produce only a partial physiological response of the endogenous ligand. For example, the response is about 10%, about 20%, about 30%, about 40%, about 50%, about 60%, about 70%, about 80%, about 90%, or about 99% of the full physiological response of an endogenous ligand.

For example, a kappa opioid receptor agonist can refer to a compound that binds to and activates a specific kappa opioid receptor type. Any pharmacologically acceptable kappa opioid agonist will function in the invention, as the underlying mechanism is dependent upon binding to and stimulating kappa opioid receptors.

There are at least five major categories of kappa opioid agonists: (1) the dynorphins, which are endogenous peptides and their derivatives; (2) the benzodiazepine derivatives, such as tifluadom; (3) the benzomorphan derivatives, such as ethylketocyclazocine, ketocyclazocine, and bremazocine; (4) the benzeneacetamide derivatives, such as U-50,488H, U-62,066E, U-69,593, CI-977, and PD 117302; and (5) the aminomethylpyridines, such as BRL 52537, BRL 52656, BRL 53114, GR89696, GR86014, and GR91272.

Non-limiting examples of kappa opioid agonists are listed herein. See, for example, U.S. Pat. 5,859,043, which is incorporate by reference herein in its entirety. In many cases the listings include references to a commercial source, a citation for the synthesis of a compound, or both.

I. L Kappa Opioid Agonists available through SmithKline Beecham Pharmaceuticals, Department of Renal Pharmacology, King of Prussia, Pa.; or Department of Biology, SmithKline Beecham Farmaceutici, Baranzate, Milan, Italy:
BRL 52537 (2S)-1-[3,4-dichlorophenylacetyl]-2-[(1-pyrrolidinylmethyl]-piperidine
BRL 52656 (2S)-1-[4-trifluoromethyl-phenyl]acetyl]-2-[(1-pyrrolidinyl)methyl]piperidine V. Vecchietti et al., J. Med. Chem., vol. 34, pp. 397-403 (1991).
BRL 53114 (-)-1-(4-trifluoromethylphenyl)-acetyl-2-(1-pyrrolidinylmethyl)-3,3-dimethyl-piperidine hydrochloride
BRL 52974 4-(1-pyrrolidinylmethyl)-5-(3,4-dichlorophenyl)acetyl-4,5,6,7-tetrahydroimidazo-[4,5-c]-pyridine
BRL 53117 1-[(3,4-dichlorophenyl)acetyl]-2-[(3-hydroxy-1-pyrrolidinyl)-methyl]-4,4-dimethylpiperidine
BRL 52974 5-[(3,4-dichlorophenyl)acetyl]-4-(1-pyrrolidinylmethyl)-4,5,6,7-tetrahydro-1H-imidazo[4,5-c]pyridine
BRL 53001 (2S)-2-(dimethylaminomethyl)-1-[(5,6,7,8-tetrahydro-5-oxo-2-naphthyl)acetyl]piperidine
2-(aminomethyl)piperidine derivatives, with incorporation of the 1-tetralon-6-yl-acetyl residue: G. Giardina et al., J. Med. Chem., vol. 37, pp. 3482-3491 (1994)
Compound (34) (2S)-2-[(dimethylamino)-methyl]-1-[(5,6,7,8-tetrahydro-5-oxo-2-naphthyl)-acetyl]-piperidine

II. Kappa Opioid Agonists available through The Upjohn Company, Kalamazoo, Mich.:
U-50,488H trans-(+/-)-3,4-dichloro-N-methyl-N-[2-(1-pyrrolidinyl)cyclohexyl]-benzeneacetamide methanesulphonate hydrate
J. Szmuszkovicz et al., J. Med. Chem., vol. 25, pp. 1125-1126 (1982); U.S. Pat. No. 4,098,904; U.S. Pat. No. 4,145,435; European Pat. No. 0 129 991 (1985); Chem. Abstr. vol. 91, no. 39003 g (1979).
U-62,066E (spiradoline) 3,4-dichloro-N-methyl-N-(3-methylene-2-oxo-8-(1-pyrrolidinyl)-1-oxaspiro(4,5)dec-7-yl)-benzeneacetamide
J. Szmuszkovicz et al., J. Med. Chem., vol. 25, pp. 1125-1126 (1982); U.S. Pat. No. 4,438,130; Chem Abstr. vol. 101, no. 54912w; German Pat. No. 3241933 (1985); Chem. Abstr. vol. 103, no. 184969b (1985).
U-69,593 (5-α,7-α,8-β-(-)-N-methyl-N-[7-(1-pyrrolidinyl)-1-oxaspiro(4,5)-dec-8-yl]benzeneacetamide
R. A. Lahti et al., Eur. J. Pharmacol., vol. 109, pp. 281-284 (1985).

III. Kappa Opioid Agonists available through Parke-Davis Research Unit, Addenbrooke’s Hospital Site, Hills Road, Cambridge, England, or Parke-Davis Research Division, Warner-Lambert Company, Ann Arbor, Mich.
CI-977 (enadoline=PD 129290) (5R)-(5-α, 7-α, 8-β)-N-methyl-N-[7-(1-pyrrolidinyl)-1-oxaspiro[4,5]dec-8-yl]-4-benzo-furanacetamide monohydrochloride
P. R. Halfpenny et al., J. Med. Chem., vol. 33, pp. 286-291 (1990); P. R. Halfpenny et al., J. Med. Chem., vol. 34, pp. 190-194 (1991).
PD 117302 (+/-)-trans-N-methyl-N-[2-(1-pyrrolidinyl)-cyclohexyl]-benzo[b]-thiophene-4-acetamide monohydrochloride
C. R. Clark et al., J. Med. Chem., vol. 31, pp. 831-836 (1988).
Derivatives of PD 117302:
   A. Compound (9) (+)-N-methyl-N-[7-(1-pyrrolidinyl)-1-oxaspiro[4.5]dec-8-yl]acenaphthene-carboxamide monohydrochloride
   P. R. Halfpenny et al., J. Med. Chem., vol. 34, pp. 190-194 (1989).
   B. Compound (17) (-)-4,5-dihydro-N-methyl-N-[7-(1-pyrrolidinyl)-1-oxaspiro[4.5]dec-8-yl]-3H-naphtho-[1,8-b,c]-thiophene-5-carboxamide-p-toluene sulfonate
   P. R. Halfpenny et al., J. Med. Chem., vol. 34, pp. 190-194 (1989).
   C. Compound (32) trans-(+/-)-N-methyl-N-[4,5-dimethoxy-2-(1-pyrrolidinyl)-cyclohexyl]-benzo[b]-thiophene-4-acetamide
   P. R. Halfpenny et al., J. Med. Chem., vol. 32, pp. 1620-1626 (1989).
   D. Compound (21) (-)-(5-β, 7-β, 8-α)-N-methyl-N- 7-(1-pyrrolidinyl)-1-oxaspiro [4.5]-dec-8-yl]-benzo[b]furan-4-acetamide monohydrochloride
   P. R. Halfpenny et al., J. Med. Chem., vol. 33, pp. 286-291 (1990).

IV. Kappa Opioid Agonists available through Zambeletti Research Laboratories, Baranzate, Milan, Italy
(2S)-1-(arylacetyl)-2-(aminomethyl)piperidine derivatives:
V. Vecchietti et al., J. Med. Chem., vol. 34, pp. 397-403 (1991); V. Vecchietti et al., J. Med. Chem., vol. 34, pp. 2624-2633 (1991).

A. Compound (14) (=BRL 52537A) (2S)-1-[3,4-dichlorophenyl-acetyl]-2-(pyrrolidin-1-yl-methyl) piperidine hydrochloride

B. Compound (21) (=BRL 52656A) (2S)-1-[[4-(trifluoromethyl)phenyl]acetyl]-2-(pyrrolidin-1-yl-methyl) piperidine hydrochloride

(1S)(aminomethyl)-2-(arylacetyl)-1,2,3,4-tetrahydroisoquinoline and heterocycle-condensed tetrahydropyridine derivatives:
V. Vecchietti et al., J. Med. Chem., vol. 34, pp. 2624-2633 (1991).
A. Compound (28)
B. Compound (30)
C. Compound (48)

V. Kappa Opioid Agonists available through The Du Pont Merck Pharmaceutical Co., Wilmington, Del.
DuP747 3,4-dichloro-N-methyl-N-(2-(pyrrolidin-1-yl)-1,2,3,4-tetrahydro-5-hydroxynaphthalen-1-yl)-benzeneacetamide
M. A. Hussain et al., Pharm. Res., vol. 9, pp. 750-752 (1992).
DuP E3800 (+/-)-trans-3,4-dichloro-N-methyl-[2-(pyrrolidine-1-yl)-6-hydroxy-1,2,3,4tetrahydronaphth-1-yl]-benzeneacetamide phosphate

V. Kappa Opioid Agonists available through Preclinical Pharmaceutical Research, and Department of Medicinal Chemistry, E. Merck, Darmstadt, Germany; or Merck-Clevenot S. A., Nogent-sur-Mame, France
EMD 60400 N-methyl-N-[(1S)-1-phenyl-2-((3S)-3-hydroxypyrrolidine-1-yl)-ethyl]-2-aminophenylacetamide 2HCl
A. Barber et al., Br. J. Pharmacol., vol. 111, pp. 843-851 (1994).
EMD 61753 R. Gottschlich et al., Chirality, vol. 6, pp. 685-689 (1994); A. Barber et al., Br. J. Pharmacol., vol. 113, pp. 1317-27 (1994).

VI. Kappa Opioid Agonists available through Glaxo Group Research Ltd., Dept. of Medicinal Chemistry and Neuropharmacology, Ware, Herefordshire, England:
1-[(3,4-dichlorophenyl)acetyl]-2-[(alkylamino)methyl]piperidine derivatives:
D. Scopes et al., J. Med. Chem., vol. 35, pp. 490-501 (1992); A. Hayes et al., Br. J. Pharmacol., vol. 101, pp. 944-948 (1990); H. Rogers et al., Br. J. Pharmacol., vol. 106, pp. 783-789 (1992).

A. Compound (10) 1-[3,4-dichlorophenyl-acetyl]-2-[1-(3-oxopyrrolidinyl)]methyl]piperidine

B. Compound (39) (=GR 45809) 8-[3,4-dichlorophenyl-acetyl]-7-(1-pyrrolidinylmethyl)-1,4-dioxa-8-azaspiro[4,5]decane

C. GR89696 methyl-4-[3,4-dichlorophenyl-acetyl]-3-(1-pyrrolidinylmethyl)-1-piperazinecarboxylate fumarate

D. GR86014 2-[(3,4-dichlorophenyl)acetyl]-1,2,3,4-tetrahydro-1-(1-pyrrolidinyl-methyl)-5isoquinolinol maleate

E. GR91272 5-[(3,4-dichlorophenyl)-acetyl]-4,5,6,7-tetrahydro-4-[(3-hydroxy-1-pyrrolidinyl)-methyl]furo 3,2-c]pyridine hydrochloride

F. GR44821 1-[(3,4-dichlorophenyl)acetyl]-2-[(3-oxo-1-pyrrolidinyl)methyl]piperidine maleate

G. GR103545 (R)-methyl-4-[(3,4-dichlorphenyl)acetyl]-3-(1-pyrrolidinyl-methyl)-1-piperazinecarboxylate fumarate

H. GR94839

I. GR85571

5-(arylacetyl)-4-[(alkylamino)methyl]furo 3,2-c]pyridines:
A. Naylor et al., J. Med. Chem., vol. 37, pp. 2138-44 (1994).
A. Compounds (16-23)
B. Compound (26) (=GR107537)
C. Compound (27)

Substituted trans-3-(decahydro- and octahydro-4a-isoquinolinyl) phenols:
D. Judd et al., J. Med. Chem., vol. 35, pp. 48-56 (1992).
A. Compound (10)
B. Compounds (11 a-d)
C. Compound (20)

VII. Kappa Opioid Agonists available through ICI Pharmaceutical, Research Department, Alderley Park, Macclesfield, Cheshire, England
G. Costello et al., Eur. J. Pharmacol, vol. 151, pp. 475-478 (1988).
ICI 204879 (R,S)-N-[2-(N-methyl-3,4-dichlorophenylacetamido)-2-(3,4-dimethyloxyphenyl)-ethyl]pyrrolidine hydrochloride
ICI 199441 (2S)-N-[2-(N-methyl-3,4-dichlorophenylacetamido)-2-phenylethyl]pyrrolidine hydrochloride
ICI 197067 (2S)-N-[2-(N-methyl-3,4-dichlorophenylacetamido)-3-methylbutyl]pyrrolidine hydrochloride
2-(3,4-dichlorophenyl)-N-[2-(1-pyrrolidinyl)ethyl] acetamide derivatives (U-50,488 derivatives):
G. Costello et al., J. Med. Chem., vol. 34, pp. 181-189 (1991).

A. Compound (8) 2-(3,4-dichlorophenyl)-N-methyl-N-[(1S)-1-phenyl-2-(1-pyrrolidinyl)ethyl] acetamide

2-(3,4dichlorophenyl)-N-methyl-N-[2-(1-pyrrolidinyl)-1-substituted-ethyl]acetamides:
J. Barlow et al., J. Med. Chem., vol. 34, pp. 3149-3158 (1991).
A. Compound (13) 2-(3,4-dichlorophenyl)-N-methyl-N-[(1S)-1-(1-methylethyl)-2-(1-pyrrolidinyl)-ethyl]acetamide
B. Compound (48) 2-(3,4-dichlorophenyl)-N-methyl-N-[(1R,S)-1-(3-aminophenyl)-2-(1-pyrrolidinyl)-ethyl] acetamide

VIII. Kappa Opioid Agonists available through Medizinische Klinik II and Institut fur Klinische Chemie, Klinikum Grosshadem, Munchen, Germany; or Humanpharmakologisches Zentrum, Boehringer Ingelheim KG, Ingelheim am Rhein, Germany
MR 2033 (+)-α-(1R,5R,9R)-5,9-dimethyl-2-(L-tetrahydrofurfuryl)-2′-hydroxy-6,7-benzomorphan
MR 2034 (-)-α-(1R, 5R,9R)-5,9-dimethyl-2-(L-tetrahydrofurfuryl)-2′-hydroxy-6,7-benzomorphan
H. Merz et al., J. Med. Chem., vol. 22, pp. 1475-1479 (1979).

IX. Kappa Opioid Agonist available through Preclinical Research, Pharmaceutical Division, Sandoz Ltd., Basel, Switzerland; or Kali-Chemie Pharma Ltd., Hannover, Germany
tifluadom (+)-(1-methyl-2,3-thienyl-carboxyl)-aminomethyl-5-(2-fluorophenyl)-H-2,3-dihydro-1,4-benzodiazepine
D. Romer et al., Life Sciences, vol. 31, pp. 1217-1220 (1982).

X. Kappa Opioid Agonist available through Centre de Recherches Roussel Uclaf, Romainville, France; or Hop. Sacre-Coeur, Universite de Montreal, Canada
RU 51599 (Niravoline)
G. Hamon et al., J. Am. Soc. Nephrol, vol. 5, p. 272A (1994).

XI. Dynorphin, Dynorphin Derivatives, and Analogs available through Sigma, RBI, ICN, and other pharmaceutical and biochemical distributors:
G. Martinka et al., Eur. J. Pharmacol., vol. 196, pp. 161-167 (1991).
A. Dynorphin A-(1-11)-NH2
B. [D-Ala3]Dyn A(1-11)-NH2
C. [Ala3] Dyn A(1-11)-NH2
F. Lung et al., J. Med. Chem., vol. 38, pp. 585-586 (1995).

XII. Kappa Opioid Agonist available through EISAI Chemical Co., Tsukuba Research Laboratories, Ibaraki, Japan:
Benzomorphans
ketocyclazocine (+)-3-(cyclopropylmethyl)-8-keto-5-(eq)-9(ax)-dimethyl-6,7-benzomorphan
bremazocine [SR-(5,7,8-β)]-N-methyl-N-[7-(1-pyrrolidinyl)-1-oxaspiro]-4,5- dec-8-yl] -4-benzofuranacetamide

XIII. Other Kappa Opioid Agonists
ethylketocyclazocine (Sterling Winthrop)
HN-11608 (Harslund Nycomed)
RP-60180 (Rorer)
TRK-820 (Toray)
R-84760

As described herein, circulating blood levels of vasopressin can be reduced by administration of a kappa opioid receptor agonist. For example, the kappa opioid receptor agonist can comprise JT09, nalfurafine, CR665, or CR845.

JT09 (C₃₈H₅₃N₇O₄) refers to an orally active and peripherally restricted KOA. JT09 increases urine output which reduces serum creatinine levels. JT09 comprises a compound of Structure (I):

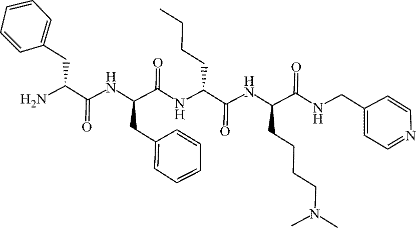 [SMILES: CCCC[C@@H](NC(=O)[C@@H](Cc1ccccc1)NC(=O)[C@H](N)Cc1ccccc1)C(=O)N[C@H](CCCCN(C)C)C(=O)NCc1ccncc1]

Nalfurafine (C₂₈H₃₂N₂O₅) refers to an orally active, potent, selective, centrally-penetrant κ-opioid receptor (KOR) agonist. Nalfurafine comprises a compound of Structure (II):

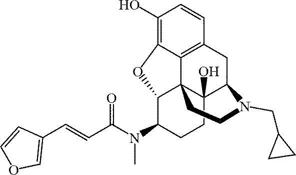

CR665 refers to a D-amino acid peptide that acts as a peripherally restricted κ-opioid receptor agonist. CR665 comprises a compound of Structure (III):

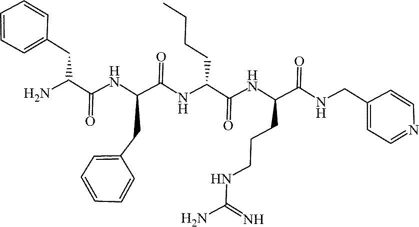

CR845 refers to a peripherally-acting, selective κ-opioid receptor agonist. CR845 comprises a compound of Structure (IV):

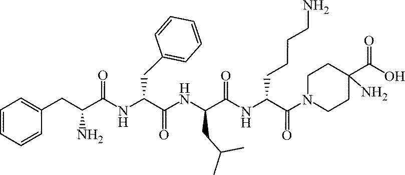 [SMILES: CC(C)C[C@@H](NC(=O)[C@@H](Cc1ccccc1)NC(=O)[C@H](N)Cc1ccccc1)C(=O)N[C@H](CCCCN)C(=O)N1CCC(N)(C(=O)O)CC1]

In embodiments, the KOA is an organic molecule. An “organic molecule” can refer to a molecule or compound that contains at least one carbon atom. The organic molecule can be an organic small molecule. A “small molecule” can refer to a chemical compound that is small enough in size so that it can readily pass through a cellular membrane unassisted. In general, a small molecule refers to chemical compounds that are not polymers, such as nucleic acids, polypeptides, or polysaccharides, although the term can encompass small polymers that can readily crossing the cellular membrane.

In embodiments, the KOA can be a peripherally restricted KOA. In embodiments, the KOA can act centrally.

In embodiments, the small molecule comprises JT09, an orally active and peripherally restricted KOA. JT09 increases urine output which reduces serum creatinine levels.

In embodiments, the small molecule comprises nalfurafine, an orally active KOA.

In embodiments, the small molecule comprises a compound of Structure (I):

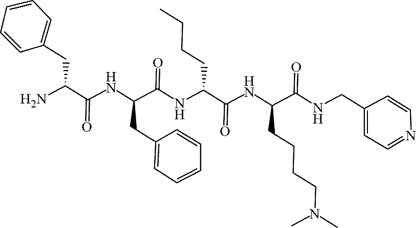 [SMILES: CCCC[C@@H](NC(=O)[C@@H](Cc1ccccc1)NC(=O)[C@H](N)Cc1ccccc1)C(=O)N[C@H](CCCCN(C)C)C(=O)NCc1ccncc1]

In embodiments, the small molecule comprises a compound of Structure (II):

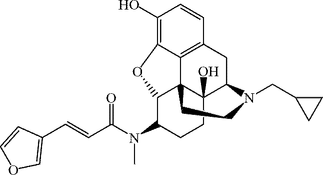

The nociceptin opioid peptide receptor (NOP) is a protein that in humans is encoded by the OPRL1 gene. The nociceptin receptor is a member of the opioid subfamily of G protein-coupled receptors whose natural ligand is the 17 amino acid neuropeptide known as nociceptin (N/OFQ). NOP shares high sequence identity with the ‘classical’ opioid receptors, but posseses little or no affinity for opioid peptides or morphine-like compounds. Likewise, classical opioid receptors possess little affinity towards NOP’s endogenous ligand, nociceptin.

Without wishing to be bound by theory, circulating blood levels of vasopressin can be reduced by administration of a nociceptin opioid peptide receptor (NOP) agonist. For example, the nociceptin opioid peptide receptor (NOP) agonist can comprise nociceptin or a peptide-conjugate thereof. Non-limiting examples of other nociceptin opoiod receptor (NOP) agonists include AT-121, Buprenorphine, BU08028, Cebranopadol, Etorphine, MCOPPB, MT-7716, Norbuprenorphine, NNC 63-0532, RO64-6198, Ro65-6570, SCH-221,510, SR-16435, and TH-030418.

Nociceptin, the classical ligand of the nociceptin opioid peptide receptor, is a 17 amino acid peptide related to the opioid class of compounds, but does not act at the classic opioid receptors, such as mu, kappa, or delta opioid receptors. The amino acid sequence of nociceptin is:

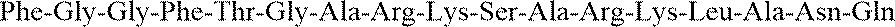

Effects of nociceptin in the CNS include: hyperalgesia/hypoalgesia, stimulation of appetite and gnawing, increased (low doses) or decreased (high doses) locomotion, impaired learning, and dysphoria. However, nociceptin also exerts important effects outside the CNS. Thus, low doses of nociceptin increase the renal excretion of water and decrease urinary sodium excretion (i.e., produces a selective water diuresis) which render this compound interesting for the treatment of hyponatremia (Daniel R. Kapusta, Life Science, 60:15-21, 1997) (U.S. Pat. No. 5,840,696). When administered centrally (i.c.v.) or at high doses peripherally (i.v. bolus or infusion), nociceptin decreases blood pressure, heart rate and peripheral sympathetic nerve activity. When administered centrally, nociceptin reduces circulating levels of vasopressin. See, for example, Kakiya, Satoshi, et al. “Role of endogenous nociceptin in the regulation of arginine vasopressin release in conscious rats.” Endocrinology 141.12 (2000): 4466-4471, which is incorporated herein by reference in its entirety.

Previous studies have demonstrated that nociceptin produces a marked increase in urine flow rate and decrease in urinary sodium excretion (i.e., an aquaretic response) when administered centrally (intracerebro-ventricularly, i.c.V.) or as an i.v. infusion in conscious rats. In the conscious rat model, the animal is chronically instrumented with catheters in the urinary bladder, the femoral artery, and the femoral vein. The animal receives a continuous i.v. infusion with isotonic saline, 50 ul/min. Urine is collected in vials in consecutive urine collection periods of 10 min each. After two control periods, the test compound is administered and fractionated urine collections (10 min periods) are continued for at least two hours. Urinary concentrations of sodium and potassium are determined by flame photometry (Instrumental Laboratory 943) using caesium as internal standard.

Other NOP agonists, such as AT-390, AT-090, AT-127, and AT-403, can increase urine output in Sprague-Dawley rats. AT-403, AT-090 and AT-127 can cause sedation and hyperphagia. Denys, Ian B., et al. “Characterization of the Cardiovascular and Renal Effects of Synthetic Nociceptin/Orphanin FQ Receptor Partial Agonists.” The FASEB Journal 32.1_supplement (2018): 568-11. Such agonists, for example, can be used in embodiments as described herein.

For example, the NOP agonist can have a structure according to Structure (V):

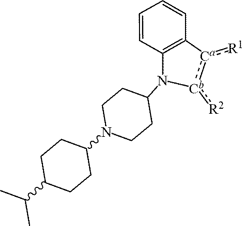

wherein

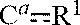

is a single bond and R¹ is CH₂—CH═O—NH—CH₃, CH₂—OH, alkyl, OH, or H; and

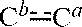

is a double bond and

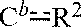

is a single bond and R² is CH₂—OH, OH, H, or alkyl; or

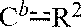

is a single bond and

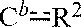

is a double bond and R² is O; Or

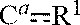

is a double bond and R¹ is O, N—OH; and

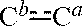

is a single bond; and

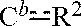

is a double bond and R² is O.

For example, the NOP agonist can be AT-403, which is a nonpeptide NOP full agonist. See, for example, Ferrari, Federica, et al. “In vitro pharmacological characterization of a novel unbiased NOP receptor-selective nonpeptide agonist AT-403.” Pharmacology research & perspectives 5.4 (2017): e00333. Accordingly, the NOP agonist can be a structure according to Structure (VI):

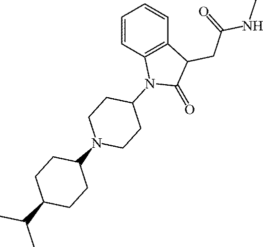

For example, the NOP agonist can be anon peptide partial agonist, such as AT-390, AT-127 and AT-090. See, for example, Ferrari, Federica, et al. “In vitro functional characterization of novel nociceptin/orphanin FQ receptor agonists in recombinant and native preparations.” European journal of pharmacology 793 (2016): 1-13. See, for example, Mercatelli, Daniela, et al. “Managing Parkinson’s disease: moving ON with NOP.” British Journal of Pharmacology 177.1 (2020): 28-47. Accordingly, the NOP agonist can be a structure according to Structure (VII):

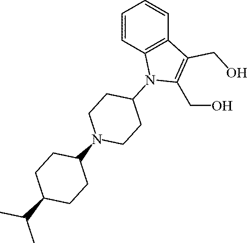

Accordingly, the NOP agonist can be a structure according to Structure (VIII):

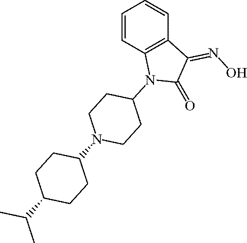

Accordingly, the NOP agonist can be a structure according to Structure (IX):

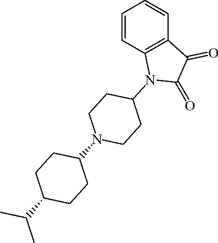

Dooley et al. (The Journal of Pharmacology and Experimental Therapeutics, 283(2):735-741, 1997) have shown that a positively charged hexapeptide having the amino acid sequence Ac-RYY(RK)(WI)(RK)-NH₂, where the brackets show allowable variation of amino acid residue, acts as a partial agonist of the nociceptin receptor ORL1. “Partial agonist” can refer to an agonist which is unable to induce maximal activation of a receptor population, regard less of the amount of drug applied (See also Intrinsic activity). A “partial agonist” can also be termed “agonist with intermediate intrinsic efficacy” in a given tissue. Moreover, a partial agonist can antagonize the effect of a full agonist that acts on the same receptor.

In embodiments, the NOP agonist can be a peptide NOP receptor partial agonist. For example, ZP120: Ac—Arg—Tyr—Tyr—Arg—Trp—Lys—Lys—Lys—Lys—Lys—Lys—Lys—NH2, is a peptide NOP receptor partial agonist that has been shown to be efficacious in lowering blood pressure in human trials. See, for example, Kantola I, Scheinin M, Gulbrandsen T, Meland N, Smerud KT. Safety, Tolerability, and Antihypertensive Effect of SER100 (also known as ZP120), an Opiate Receptor-Like 1 (ORL-1) Partial Agonist, in Patients With Isolated Systolic Hypertension. Clin Pharmacol Drug Dev. 2017 Nov;6(6):584-591. doi: 10.1002/cpdd.330. Epub 2016 Dec 29. PMID: 28032481.

Without wishing to be bound by theory, circulating blood levels of vasopressin can be reduced by administering, such as centrally, to a subject nociceptin or a peptide conjugate thereof. For example, such peptide conjugates can be found in U.S. Pat. 7,244,701 and U.S. Pat. 7,550,425, each of which are incorporated by reference herein in their entireties.

Embodiments as described herein, whether a kappa opioid receptor agonist or a nociceptin opioid peptide receptor agonist can be provided as a pharmaceutically acceptable salt. The term “pharmaceutically acceptable” can refer to salts or chelating agents are acceptable from a toxicity viewpoint. The term “pharmaceutically acceptable salt” can refer to ammonium salts, alkali metal salts such as potassium and sodium (including mono, di- and tri-sodium) salts, alkaline earth metal salts such as calcium and magnesium salts, salts with organic bases such as dicyclohexylamine salts, N-methyl-D-glucamine, and salts with amino acids such as arginine, lysine, and so forth.

The term “salt”, as used herein, denotes acidic and/or basic salts, formed with inorganic or organic acids and/or bases, for example basic salts. While pharmaceutically acceptable salts can be used when employing the compounds of the invention as medicaments, other salts can be used as well, for example, in processing these compounds, or for non-medicament-type uses. Salts of these compounds can be prepared by art-recognized techniques. Examples of such pharmaceutically acceptable salts include, but are not limited to, inorganic and organic acid addition salts, such as hydrochloride, sulphates, nitrates or phosphates and acetates, trifluoroacetates, propionates, succinates, benzoates, citrates, tartrates, fumarates, maleates, methane-sulfonates, isothionates, theophylline acetates, salicylates, respectively, or the like. Lower alkyl quaternary ammonium salts and the like are suitable, as well. “Pharmaceutically acceptable anions” as used herein includes the group consisting of CH₃COO^{—}, CF₃COO^{—}, Cl^{—}, SO₃ ^{2—}, maleate and oleate.

Embodiments as described herein can be administered to a subject in the form of a pharmaceutical compositions suitable for the intended route of administration. Such compositions can comprise, for example, the active ingredient, such as the kappa opioid receptor agonist, and a pharmaceutically acceptable carrier. Such compositions can be in a form adapted to oral, subcutaneous, parenteral (intravenous, intraperitoneal), intramuscular, rectal, epidural, intratracheal, intranasal, dermal, vaginal, buccal, ocularly, or pulmonary administration, for example, in a form adapted for administration by a peripheral route, or is suitable for oral administration or suitable for parenteral administration. Other routes of administration are subcutaneous, intraperitoneal and intravenous, and such compositions can be prepared in a manner well-known to the person skilled in the art, e.g., as described in “Remington’s Pharmaceutical Sciences”, 17. Ed. Alfonso R. Gennaro (Ed.), Mark Publishing Company, Easton, Pa., U.S.A., 1985 and more recent editions and in the monographs in the “Drugs and the Pharmaceutical Sciences” series, Marcel Dekker. The compositions can appear in conventional forms, for example, solutions and suspensions for injection, capsules and tablets, such as in the form of enteric formulations, e.g. as disclosed in U.S. Pat. No. 5,350,741, for oral administration.

The pharmaceutical carrier or diluent employed can be a conventional solid or liquid carrier. Examples of solid carriers are lactose, terra alba, sucrose, cyclodextrin, talc, gelatin, agar, pectin, acacia, magnesium stearate, stearic acid or lower alkyl ethers of cellulose. Examples of liquid carriers are syrup, peanut oil, olive oil, phospholipids, fatty acids, fatty acid amines, polyoxyethylene and water. Similarly, the carrier or diluent can include any sustained release material known in the art, such as glyceryl monostearate or glyceryl distearate, alone or mixed with a wax.

When a solid carrier is used for oral administration, the preparation can be tabletted, placed in a hard gelatin capsule in powder or pellet form or it can be in the form of a troche or lozenge. The amount of solid carrier will vary widely but will be from about 25 mg to about 1 g.

When a liquid carrier is used, the preparation can be in the form of a syrup, emulsion, soft gelatin capsule or sterile injectable liquid such as an aqueous or non-aqueous liquid suspension or solution.

The composition can also be in a form suited for local or systemic injection or infusion and can, as such, be formulated with sterile water or an isotonic saline or glucose solution. The compositions can be in a form adapted for peripheral administration only, with the exception of centrally administrable forms. The compositions can be in a form adapted for central administration.

The compositions can be sterilized by conventional sterilization techniques which are well known in the art. The resulting aqueous solutions can be packaged for use or filtered under aseptic conditions and lyophilized, the lyophilized preparation being combined with the sterile aqueous solution prior to administration. The composition can contain pharmaceutically acceptable auxiliary substances as required to approximate physiological conditions, such as buffering agents, tonicity adjusting agents and the like, for instance sodium acetate, sodium lactate, sodium chloride, potassium chloride, calcium chloride, etc.

In some embodiments, the compounds of the invention are present in a pharmaceutical composition comprising a pharmaceutically acceptable carrier. According to the invention, a pharmaceutically acceptable carrier can comprise any solvents, dispersion media, coatings, antibacterial and antifungal agents, isotonic and absorption delaying agents, and the like, compatible with pharmaceutical administration. The use of such media and agents for pharmaceutically active substances is well known in the art. Any conventional media or agent that is compatible with the active compound can be used. Supplementary active compounds can also be incorporated into the compositions.

Multi-dose formulations can be prepared as a solution of a compound of the invention in sterile, isotonic saline, stored in capped vials, and if necessary a preservative is added (e.g. benzoates). Fixed dose formulations can be prepared as a solution of the compound in sterile, isotonic saline, stored in glass ampoules, and if necessary filled with an inert gas. Each dose of the compound is stored dry in ampoules or capped vials, if necessary filled with inert gas. The multi-dose formulation demands the highest degree of stability of the compound. When the stability of the compound is low fixed dose formulations can be used. For nasal administration, the preparation can contain a compound of the invention dissolved or suspended in a liquid carrier, for example, an aqueous carrier, for aerosol application. The carrier can contain additives such as solubilizing agents, e.g., propylene glycol, surfactants such as bile acid salts or polyoxyethylene higher alcohol ethers, absorption enhancers such as lecithin (phosphatidylcholine) or cyclodextrin, or preservatives such as parabines.

In embodiments, the kappa opioid receptor agonist or nociceptin opioid peptide receptor agonist can be administered to the subject in a therapeutically effective amount. As used herein, the term “therapeutically effective amount” can refer to that amount of the therapeutic agent sufficient to realize a biological effect, such as treating a disease or disorder such as polycystic kidney disease.

A “therapeutically effective dose” can refer to that amount of the therapeutic agent sufficient to result in amelioration of symptoms, e.g., treatment, healing, prevention or amelioration of the relevant medical condition, or an increase in rate of treatment, healing, prevention or amelioration of such conditions. When applied to an individual active ingredient administered alone, a therapeutically effective dose can refer to that ingredient alone. When applied to a combination, a therapeutically effective dose can refer to combined amounts of the active ingredients that result in the therapeutic effect, whether administered in combination, serially or simultaneously.

The term “treating” can refer to partially or completely alleviating, ameliorating, improving, relieving, delaying onset of, inhibiting progression of, reducing severity of, and/or reducing incidence of one or more symptoms, features, or clinical manifestations of a disease, disorder, and/or condition, such as polycystic kidney disease. For example, “treating” polycystic kidney disease can refer to (or be indicated by) decreasing circulating blood levels of vasopressin, maintaining glomerular filtration rate, reducing kidney fibrosis, decreasing blood urea levels, decreasing cysts number, decreasing cysts size, decreasing serum creatinine levels, or a combination thereof. Treatment can be administered to a subject who does not exhibit signs of a disease, disorder, and/or condition (e.g., prior to an identifiable disease, disorder, and/or condition), and/or to a subject who exhibits only early signs of a disease, disorder, and/or condition for the purpose of decreasing the risk of developing pathology associated with the disease, disorder, and/or condition.

In embodiments, a therapeutically effective amount, such as a therapeutically effective amount of a kappa opioid receptor agonist or nociceptin opioid peptide receptor agonist, can comprise a dose of about 0.005 mg/kg to about 1000 mg/kg. In some embodiments, a therapeutically effective amount can comprise a dose of about 0.005 mg/kg to about 10 mg/kg. In some embodiments, a therapeutically effective amount can comprise a dose of about 0.25 mg/kg to about 2 mg/kg. In some embodiments, the therapeutically effective amount is at least about 0.001 mg/kg at least about 0.0025 mg/kg, at least about 0.005 mg/kg, at least about 0.01 mg/kg, at least about 0.1 mg/kg body weight, at least about 0.25 mg/kg body weight, at least about 0.5 mg/kg body weight, at least about 0.75 mg/kg body weight, at least about 1 mg/kg body weight, at least about 2 mg/kg body weight, at least about 3 mg/kg body weight, at least about 4 mg/kg body weight, at least about 5 mg/kg body weight, at least about 6 mg/kg body weight, at least about 7 mg/kg body weight, at least about 8 mg/kg body weight, at least about 9 mg/kg body weight, at least about 10 mg/kg body weight, at least about 15 mg/kg body weight, at least about 20 mg/kg body weight, at least about 25 mg/kg body weight, at least about 30 mg/kg body weight, at least about 40 mg/kg body weight, at least about 50 mg/kg body weight, at least about 75 mg/kg body weight, at least about 100 mg/kg body weight, at least about 200 mg/kg body weight, at least about 250 mg/kg body weight, at least about 300 mg/kg body weight, at least about 3500 mg/kg body weight, at least about 400 mg/kg body weight, at least about 450 mg/kg body weight, at least about 500 mg/kg body weight, at least about 550 mg/kg body weight, at least about 600 mg/kg body weight, at least about 650 mg/kg body weight, at least about 700 mg/kg body weight, at least about 750 mg/kg body weight, at least about 800 mg/kg body weight, at least about 900 mg/kg body weight, or at least about 1000 mg/kg body weight. However, the skilled artisan will recognize that the dosage can vary depending upon known factors such as the pharmacodynamic characteristics of the active ingredient and its mode and route of administration; time of administration of active ingredient; age, sex, health and weight of the recipient; nature and extent of symptoms; kind of concurrent treatment, frequency of treatment and the biological effect desired; and rate of excretion.

As described herein, embodiments herein can be formulated into a pharmaceutical composition to be compatible with its intended route of administration. Non-limiting examples of routes of administration include oral or parenteral, e.g., intravenous, intradermal, subcutaneous, inhalation, intranasal, transdermal (topical), transmucosal, and rectal administration.

Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EM™ (BASF, Parsippany, N.J.) or phosphate buffered saline (PBS). The composition can be sterile and can be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, a pharmaceutically acceptable polyol like glycerol, propylene glycol, liquid polyetheylene glycol, and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, and thimerosal. In many cases, it can be useful to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the compound in the required amount in an appropriate solvent with one or a combination of ingredients enumerated herein, as required, followed by filtered sterilization. Dispersions are prepared by incorporating the active compound into a sterile vehicle which contains a basic dispersion medium and the required other ingredients from those enumerated herein. In the case of sterile powders for the preparation of sterile injectable solutions, examples of useful preparation methods are vacuum drying and freeze-drying which yields a powder of the active ingredient plus any additional ingredient from a previously sterile-filtered solution thereof.

Oral compositions can include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Oral formula of the drug can be administered once a day, twice a day, three times a day, or four times a day, for example, depending on the half-life of the drug.

Pharmaceutically compatible binding agents, and/or adjuvant materials can be included as part of the composition administered to a subject. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or sterotes; a glidant such as colloidal silicon dioxide; a sweetening agent such as sucrose or saccharin; or a flavoring agent such as peppermint, methyl salicylate, or orange flavoring.

Systemic administration can also be by transmucosal or transdermal means. For transmucosal or transdermal administration, penetrants appropriate to the barrier to be permeated are used in the formulation. Such penetrants are known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as known in the art.

In embodiments, administering can comprise the placement of a pharmaceutical composition, such as a composition comprising a kappa opioid receptor agonist, into a subject by a method or route which results in at least partial localization of the composition at a site such that the effect is produced.

For example, the kappa opioid receptor agonist or nociceptin opioid peptide receptor agonist can be administered by bolus injection or by infusion. A bolus injection can refer to a route of administration in which a syrine is connected to the IV access device and the medication is injected directly into the subject. The term “infusion” can refer to an intravascular injection.

Embodiments as described herein can be administered to a subject one time (e.g., as a single injection, bolus, or deposition). Alternatively, administration can be once or twice daily to a subject for a period of time, such as from about 2 weeks to about 28 days. It can also be administered once or twice daily to a subject for period of 1, 2, 3, 4, 5, 6, 7, 8, 9, 10, 11, 12 times per year, or a combination thereof.

In embodiments, compositions as described herein can be administered to a subject chronically. “Chronic administration” can refer to administration of the kappa opioid receptor agonist or nociceptin opioid peptide receptor agonist in a continuous manner, such as to maintain the therapeutic effect (activity) over a prolonged period of time.

In embodiments, the term “subject” or “patient” can refer to any organism to which aspects of the invention can be administered, e.g., for experimental, diagnostic, prophylactic, and/or therapeutic purposes. Typical subjects to which compounds described herein can be administered will be mammals, for example primates, especially humans. For veterinary applications, a wide variety of subjects will be suitable, e.g., livestock such as cattle, sheep, goats, cows, swine, and the like; poultry such as chickens, ducks, geese, turkeys, and the like; and domesticated animals, for example, pets such as dogs and cats. For diagnostic or research applications, a wide variety of mammals will be suitable subjects, including rodents (e.g., mice, rats, hamsters), rabbits, primates, and swine such as inbred pigs and the like. The term “living subject” refers to a subject noted herein or another organism that is alive. The term “living subject” refers to the entire subject or organism and not just a part excised (e.g., a liver or other organ) from the living subject. The terms “subject”, “individual”, and “patient” can be used interchangeably.

### EXAMPLES

Examples are provided herein to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1 - Experimental Design

### Study 1: Metabolic Cage Study and Cysts Growth Evaluation

Pkd1 RC/RC mice were used as the polycystic kidney disease model. 12 weeks old mice were given a KOA treatment (JT-09 or nalfurafine) or a vehicle at a dose of 5 mg/kg for JT-09 or 100 µg/kg for nalfurafine daily for 4 or 8 weeks.

Group 1: Pkd1 RC/RC mice + treatment (JT09 or nalfurafine) oral gavage (5 mg/kg for JT-09 or 100 µg/kg for nalfurafine) daily. n=24

Group 2: Pkd1 RC/RC mice + vehicle oral gavage daily. n=24

At 16 weeks old, 12 mice from both groups 1 and 2 were selected. These mice were each placed in a metabolic cage to collect urine for three 24-hour periods (a total of 72 hours). Each mouse was placed in a separate metabolic cage, as opposed to all mice in a single metabolic cage. Daily water intake for each mouse is also recorded. Mice were then deep anesthetized, and blood was be taken from their artery. Mice were then sacrificed. Both kidneys were perfused with PBS and fixed in neutral buffered formalin. Kidneys were processed for paraffin embedding and sectioning. H&E and picrosirus stain were performed on each section. Cystic index including cysts size and number were evaluated by a pathologist. Fibrosis was evaluated by the pathologist as well; in addition, a fibrotic score was given. The serum creatinine and blood urea level in the blood was then measured.

At 20 weeks old, the other set of 12 mice in both groups was then sacrificed and the same experiment was be performed as described herein.

### Study 2: Glomerular Filtration Rate (GFR) Evaluation

Group 1: Pkd1 RC/RC mice + treatment (JT09 or nalfurafine) oral gavage (5 mg/kg for JT-09 or 100 µg/kg for nalfurafine) daily. n=24

Group 2: Pkd1 RC/RC mice + vehicle oral gavage daily. n=24

At 16 weeks old, 12 mice from each group were anesthetized. The right jugular vein was catheterized for inulin infusion, and the bladder was catheterized for urine collection. GFR was evaluated based on the excretion rate of FITC-sinistrin.

At 20 weeks old, the same GFR measure was be repeated on the other 12 mice from each group.

### Results

Without wishing to be bound by theory, KOA treatment, such as JT09 or nalfurafine treatment, will decrease cyst size and numbers, reduce fibrosis area in the kidney, decrease serum creatinine and blood urea levels. Further, JT09 or nalfurafine treatment will improve GFR in mice with PKD. The difference in the parameters between the JT09 and control group will increase at 20 weeks, compared to the data collected in 16 weeks.

### Example 2 - Use of Kappa Opioid Receptor Agonist to Treat Polycystic Kidney Disease

Administration of a kappa opioid receptor agonist (KOA) to animals or man causes an increase in urine output (diuresis) often associated with a decrease in urinary sodium/potassium excretion; this pattern of changes in water and electrolytes is referred to as a water diuresis (aquaresis). Research in our laboratory has demonstrated that both intravenous bolus injection and infusion of different KOAs are able to produce a significant water diuresis in rats and mice. Polycystic kidney disease (PKD) is a pathology characterized with variable size cysts growing in the kidneys, which replace normal nephron tissue, structure, and function. Water and electrolyte imbalance, including fluid overload and hyponatremia, can occur in patients with PKD. Progression of the disease leads to chronic renal failure and need for dialysis and kidney transplantation. Tolvaptan is the only drug that has shown efficacy in slowing down the progress of PKD, and its mechanism of action is to act in the collecting duct of the kidneys to block vasopressin type 2 (V2) receptors to cause water diuresis. KOAs also produce water diuresis but by a mechanism different than Tolvaptan. Therefore, without wishing to be bound by theory, KOA can be used for treatment of PKD. JT09 is a newly synthesized orally active KOA, which will be efficacious in chronic management of PKD. Nalfurafine is also an orally active KOA which will be efficacious in chronic management of PKD. Here, we claim, for example, that: 1) oral administration of a kappa opioid receptor agonist, such as JT09 or nalfurafine, will normalize fluid and electrolyte balance in the types of PKD, including autosomal dominant PKD1 and PKD2 (ADPKD); autosomal recessive PKD; and 2) oral administration of JT09 or nalfurafine will slow down the pathology progression of the types of PKD, including maintaining glomerular filtration rate, decreasing cysts number and size, and decreasing serum creatinine levels.

Kappa opioids, including the orally active kappa agonists, JT09 and nalfurafine, can be used for chronic management of patients who have been diagnosed with PKD.

The V2 vasopressin receptor antagonist, tolvaptan, has recently been approved by the FDA for treatment for ADPKD. In PKD, elevated levels of vasopressin are a major contributor to cyst growth by stimulating cell proliferation and fluid secretion. JT09 and nalfurafine are orally active kappa opioids that act to decrease circulating blood levels of vasopressin by inhibiting this hormone’s release from the brain. Without wishing to be bound by theory, decreasing circulating blood levels of vasopressin, JT09 and nalfurafine (and other kappa opioids, such as CR665 or CR845), will be new and highly efficacious drugs for treatment of PKD.

Without wishing to be bound by theory kappa-opioid receptor agonist treatment, such as JT09 or nalfurafine treatment (such as chronic oral dosing), will decrease cysts size in PKD mice as compared with age matched PKD mice treated with placebo. We have obtained a colony of ADPKDl mice which are an established model for studies of PKD pathology.

### Example 3 - Using Kappa Opiod Agonists to Treat Polycystic Disease by Diuresis and Attenuating Inflammation

Polycystic kidney disease (PKD) affects one person in every 400 to 1000 people in the U.S., which is associated with substantial mortality. It results in chronic kidney disease (CKD) and end-stage renal disease (ESRD), which require dialysis or kidney transplantation to survive, and have been shown to have 30% higher prevalence in veterans than in general population. Patients with PKD develop fluid-filled cysts and interstitial fibrosis in both kidneys, which cause progressive loss of renal function demonstrated as gradual decline of glomerular filtration rate (GFR). There is no cure for PKD, and currently only one treatment is available to delay progression: tolvaptan, a vasopressin V2 receptor antagonist. This drug is expensive and indicated only in some patients who are at high risk for progression. The efficacy of tolvaptan proves that an active vasopressin pathway plays a critical role in this disease progression. However, the drug carries risk of severe hepatic toxicity, and patients treated with tolvaptan are suffering serious side effects, such as severe thirst and polyuria due to inhibition of V2 receptor in the distal nephrons. Research from our laboratory and others has demonstrated that administration of kappa opioid receptor agonists (KOAs) produce a water diuresis similar to tolvaptan, but by acting in the central nervous system to inhibit the release/secretion of vasopressin into the systemic circulation. Our data show that oral gavage of nalfurafine, a KOA, produces a marked increase in urine output in wide type mice. Nalfurafine is highly selective for kappa receptors and does not produce any apparent behavioral or adverse effects. In addition to their inhibitory effect on vasopressin secretion and subsequent diuretic effect, KOAs can produce an anti-inflammatory micro-environment at the cellular level, which protects cardiac tissue from fibrosis. Thus, without wishing to be bound by theory, attenuating circulating blood levels of vasopressin and its subsequent actions in the kidneys, KOAs will be superior to tolvaptan in attenuating fluid filled cysts and fibrosis formation in PKD.

PKD is a monogenetic disorder with two typical types: autosomal dominant PKD (ADPKD) and autosomal recessive PKD (ARPKD). ADPKD accounts for 95% of PKD patients and ARPKD is very rare. We have obtained a highly clinically relevant mouse model of ADPKD from investigators, who are internationally recognized for their contributions to PKD research. We have successfully bred this colony of mice in our animal care facility. Therefore, we plan to use these mice to carry out studies which will investigate the following two Specific Aims:

Specific Aim 1: Nalfurafine administration will decrease formation and enlargement of cysts in the kidneys of mice with ADPKD. We will give Nalfurafine or placebo to mice with ADPKD for 1 or 2 months and determine whether Nalfurafine delays the decline of GFR, decreases cysts number and size in the kidneys, and down-regulates the critical signaling pathways which regulate cyst fluid production.

Specific Aim 2: Nalfurafine will decrease inflammation and interstitial fibrosis in kidneys of mice with ADPKD. We will give Nalfurafine or placebo to mice with ADPKD for 1 or 2 months and determine whether Nalfurafine decreases extracellular matrix deposition, and macrophages infiltration and pro-inflammatory cytokines production in the kidneys of mice with ADPKD.

The current study is highly innovative, supported by a provisional patent application filed recently. The results from this study will lead to development of effective therapies to treat ADPKD, and bring improvement of the current guideline of PKD treatment. Nalfurafine has been approved for clinical use in Japan as an antipruritic drug in patients with CKD undergoing hemodialysis, thus demonstrating its safety in humans. My collaboration with Nephrologists team in the PKD special clinic at our institute enables a quick translation of the findings of current study to patients with PKD.

### Example 4 - Identification of a New Drug to Treat Polycystic Kidney Disease

### Abstract

PKD affects approximately 600,000 people in the U.S., which cost the military and other federal program more than $20 billion annually for related therapies, tests, dialysis and transplantation. Patients with PKD develop fluid-filled cysts and interstitial fibrosis in both kidneys, which ultimately result in chronic kidney disease (CKD) and end-stage renal disease (ESRD), requiring dialysis or kidney transplantation to survive and is associated with substantial mortality. Currently there is only one therapy available to delay progression of PKD: tolvaptan, a vasopressin V2 receptor antagonist. Patients treated with tolvaptan have suffered serious side effects and it is extremely expensive. Therefore, this drug is indicated only in some patients who are at high risk for progression. Nalfurafine is an orally active biased kappa opioid agonist (KOA). It does not produce CNS-mediated side effects. Our data have demonstrated that nalfurafine produces diuresis, such as by inhibiting vasopressin release in the brain. KOA have been proven to decrease inflammation and inhibit cyclic adenosine monophosphate (cAMP) signaling pathway. Without wishing to be bound by theory, oral administration of nalfurafine reduces renal inflammation and proliferation of tubular cells, and improves renal function in mice with ADPKD. We will use the highly clinically relevant animal model Pkd1^{RC/RC} mice to carry out studies and validate the following two specific aims:
1. Validate the effects of nalfurafine on reducing cystogenesis during the early stage of ADPKD. We will determine whether Nalfurafine attenuates proliferation of tubular epithelial cells, decreases total kidney volume, or decreases inflammatory markers in the early phase of disease course.
2. Validate the effects of nalfurafine on reducing the severity of fibrosis and the deline of renal function (GFR) in the later stages of ADPKD.

We will use an innovative technique to measure GFR on conscious free-moving mice to validate whether nalfurafine treatment delays the decline of GFR in mice with PKD. We will also validate whether nalfurafine decreases the kidney fibrosis in mice with ADPKD. The results will be compared with tolvaptan to validate whether nalfurafine brings more benefits than tolvaptan. The findings of this study can provide extremely valuable information to current management of ADPKD. Nalfurafine is in use in Japan, demonstrating safety, and is available commercially. The results from this study can provide direct evidence and rationale to move towards organizing a clinical trial using nalfurafine to treat ADPKD.

### 1. Specific Aims

Polycystic kidney disease (PKD) affects approximately 600,000 people in the U.S.¹ and results in a cost of more than $20 billion annually for associated therapies, tests, dialysis and transplantation. Patients with PKD develop fluid-filled cysts and interstitial fibrosis in both kidneys, which cause progressive loss of renal function demonstrated as gradual decline of glomerular filtration rate (GFR).² This results in chronic kidney disease (CKD) and end-stage renal disease (ESRD), which require dialysis or kidney transplantation to survive and are associated with substantial mortality. Currently there is no cure for PKD. The current standard of care, and the only therapy available to delay disease progression, is tolvaptan, a vasopressin V2 receptor antagonist.³ At this time, this drug is considered only for some patients who are at high risk for disease progression. Some factors that can lead to reduced compliance in use of this drug is the relatively high cost and the undesirable side effects, such as severe thirst, polyuria and hepatic toxicity. Therefore, alternative treatment options are needed in the management of PKD and this remains an area of unmet need for this patient population.

Nalfurafine is an orally active G-protein-biased kappa opioid agonist (KOA), and an approved treatment in Japan. Nalfurafine does not produce the CNS-mediated side effects that one sees with traditional opioids, such as nausea/vomiting, sedation, respiratory depression, abuse potential, addiction or euphoria⁴, and a one year post-marketing surveillance study demonstrate that it does not cause extreme thirst nor polyuria.⁵ Therefore, nalfurafine is significantly more tolerable than tolvaptan.

Nalfurafine is able to target multiple pathological pathways of PKD. During the earlier stages of PKD progression, the onset of inflammation appears in the kidneys prior to the detection of cysts⁶, which indicates that preventing inflammation is a logical course of therapy in PKD. KOAs are able to produce an anti-inflammatory microenvironment and prevent fibrosis. Importantly, KOAs also inhibit the production of cyclic adenosine monophosphate (cAMP)⁷, which stimulates the fluid secretion and cell growth that produce the cysts. Finally, KOAs act on the central nervous system to inhibit the release and secretion of vasopressin into the systemic circulation. Together, these benefits will bring a significant improvement in renal function in PKD.

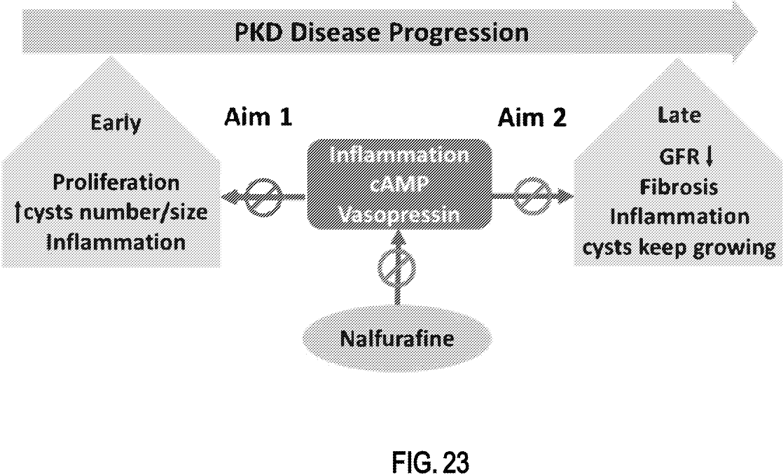

As nalfurafine has a mechanism of action that targets multiple pathological pathways of PKD, without wishing to be bound by theory, oral administration of nalfurafine reduces renal inflammation and proliferation of tubular epithelial cells, and improves renal function in mice with autosomal dominant PKD (ADPKD). As depicted in FIG. 23, we will carry out studies to test the following two specific aims:

Specific Aim 1: Validate the effects of nalfurafine on reducing cystogenesis during the early stage of ADPKD. We will validate whether nalfurafine attenuates proliferation of tubular epithelial cells and decreases total kidney volume. Additionally, we will validate whether nalfurafine decreases inflammatory markers which are elevated in the early phase of the disease course. Finally, we will compare the results between nalfurafine and tolvaptan treatments and determine whether nalfurafine demonstrates more benefits to mice with ADPKD than tolvaptan.

Specific Aim 2: Validate the effects of nalfurafine on reducing the severity of fibrosis and the deline of renal function (GFR) in the later stages of ADPKD. We will use an innovative, non-invasive and non-terminal technique to measure GFR in ADPKD mice and validate whether nalfurafine delays the decline of GFR. We will also determine whether nalfurafine decreases fibrosis in the kidneys of mice with ADPKD, and whether the extent of reduction in fibrosis is more than that of tolvaptan.

### 2. Significance

PKD is associated with significant morbidity and mortality. This genetic disease results in the continuous growth of fluid-filled cysts that originate from the renal tubules. The cysts cause apoptosis/necrosis of the nephrons, and thus interstitial fibrosis within the kidney, resulting in a continuous decline in renal function.

### Chronic Inflammation in ADPKD and KOA

In ADPKD, fluid-filled cysts which exist in both the cortex and medulla compress the surrounding glomeruli and tubules, impeding both urine and blood flow. This leads to chronic inflammation and hypoxia to the normal functioning nephrons, causing further damage to the kidneys independent of the genetically-motivated cyst formation.⁸ Withough wishing to be bound by theory, KOAs can produce an anti-inflammatory micro-environment at the cellular level by inhibiting inflammatory cytokine production and immune cell activity in the cardiac tissue.^{9,10} Nalfurafine has been proven to decrease inflammation in atopic dermatitis and pruritus, demonstrated by a reduced infiltration of CD4+ T cells and CD8+ T cells in the affected skin.¹¹ Therefore, without wishing to be bound by theory, nalfurafine will attenuate chronic inflammation in the kidneys of ADPKD.

### Proliferation of Tubular Epithelial Cells and cAMP Pathways in ADPKD

In renal tubular epithelial cells, the cyclic adenosine monophosphate (cAMP) signaling pathway is pivotal in mediating cell proliferation and fluid secretion. By blocking V2 receptors, administration of tolvaptan has been proven to down-regulate this pathway in the kidney.¹² Interestingly, tissue levels of cAMP are increased in numerous animal models of PKD not only in kidneys but also in cholangiocytes, vascular smooth muscle (VSM) cells, and choroid plexus.^{13,14} KOAs are well known to inhibit cAMP and kappa receptors are expressed in multiple organs including the kidneys, vasculature, and brain.^{15,16} Thus, via inhibiting cAMP at different sites within multiple organs, KOAs are able to tackle more targets in PKD than tolvaptan.

### Vasopressin in PKD and KOA

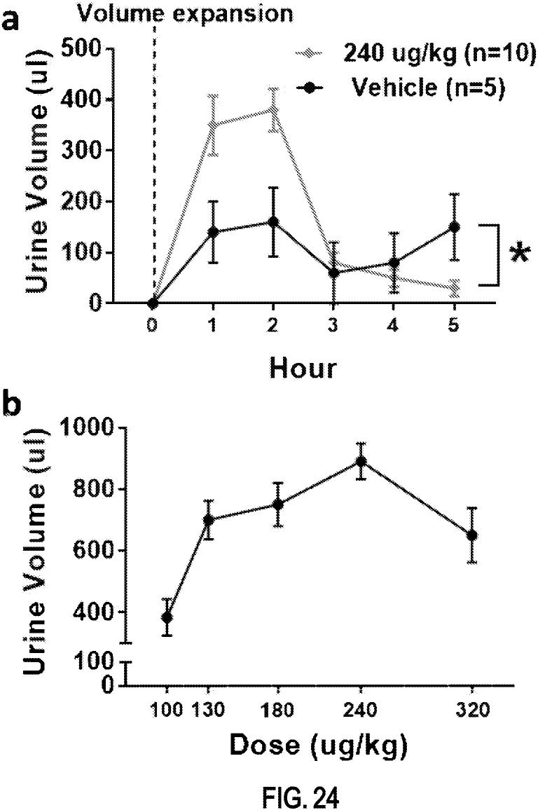

ADPKD patients have an elevated level of vasopressin. Based on this observation, tolvaptan was discovered and later approved by its mechanism of action of blocking V2 receptors on the distal tubules. However, this also leads to the side effect of polyuria and severe thirst. Together, these data indicate that vasopressin pathway is critical in PKD, but completed blockage of V2 receptors is not the best strategy to treat PKD. KOAs have been demonstrated to produce an increase in urine output similar to tolvaptan; however, KOA produce diuresis indirectly by acting in the central nervous system to inhibit the release/secretion of vasopressin (antidiuretic hormone) into the systemic circulation.¹⁷ Data from our laboratory have shown that in acute studies, oral administration of nalfurafine can lead to a significant increase in urine output within the 3 hours after dosing in mice with PKD, compared with vehicle treatment in the same strain of mice (FIG. 24, panel a). Also, this response of increase in urine output is dose dependent (FIG. 24, panel b). However, none of the clinical trials of nalfurafine have reported polyuria as a side effect. Together, these data indicate that a pronounced diuretic effect can occur when the drug is first given, but with continued use the body is able to adjust the urine output and water intake to a physiologically acceptable level. Therefore, the long-term effects by nalfurafine on urine output can be limited unlike tolvaptan.

### 3. Innovation

This example will identify nalfurafine as a new treatment for ADPKD, which can be more effective and with fewer side effects compared to the current standard of care. To achieve this goal, a preclinical study will validate 1) the impact nalfurafine has on the inflammation and proliferation in the kidneys in the early phase of ADPKD; and 2) the benefits that nalfurafine brings to renal functional improvement in the later phase of ADPKD.

1) New concepts: Kappa opioid receptors are well studied in the field of pain management, but very little attention is given to the diuretic effects they cause. KOAs have been shown to have significant negative impacts upon the CNS, such as addiction, dependence and dysphoria. In contrast, nalfurafine does not have these devastating CNS side effects. The worst side effect is insomnia, shown in approximately 3% of patients by a post marketing analysis of 3,762 patients that underwent this treatment.⁵ The well documented safety of nalfurafine enables us to further study its beneficial pharmacological effects. In the PKD field, research has been focused on classical diuretics and antihypertensives. Studies have not addressed the application of kappa opioid receptors to this field. Showing a link between nalfurafine and the effects of anti-inflammation, inhibiting cAMP and vasopressin can make this drug a candidate for the treatment of PKD and also be transformative to the field of research.

2) Translation: The findings of this study can provide valuable information to current management of ADPKD. The results from this study can provide direct evidence and rationale to move towards organizing a clinical trial using nalfurafine orally to treat ADPKD. Nalfurafine is in use in Japan, demonstrating safety, and is available commercially. We have already established a close collaboration with the nephrologists in the PKD special clinic, thus we have access to PKD patients. Nalfurafine can be a groundbreaking treatment for the types of PKD and a vast improvement over the current standard of care.

3) New technology used to provide GFR information in PKD preclinical research field: The information regarding GFR decline patterns in preclinical PKD models are rare, due to the technical difficulty of traditional GFR measurements and the inadequate numbers_of PKD animals. We will use an innovative technology to measure GFR transdermally on mice. It is a_non-invasive procedure, measured on conscious free-moving animals, and allow longitudinal GFR measurement on the same animal. The results from our studies will provide accurate_information about the GFR of the most clinically relevant model Pkd1^{RC/RC} mice.

### 4. Approach

Animals and PKD progression: We have obtained Pkd1^{RC/RC} mice and have successfully bred this colony. Unlike other fast_progressive animal models, this strain of ADPKD mice develop cysts progressively as they age,¹⁸ which_closely mimics the disease course in humans. From the age of 4 months to 6 months, the number and size of cysts surge, causing an increase of total kidney volume. From the age of 9 months to 12 months, the cysts are large and numerous, and fibrosis develops rapidly to replace normal kidney tissue. Therefore, we validated the efficacy of nalfurafine in treating PKD at 3-6-months, which we referred to as the ‘early phase’, and 9-12-months, which we refer to as the ‘later phase’ of PKD in our animal model.

Drug dose: On the dose response curve in FIG. 24, panel b, we gave Pkd1^{RC/RC} mice nalfurafine at the doses of 100, 130, 180, 240, and 320 ug/kg, and the greatest increase in 5-hour urine output was observed at the dose of 240 ug/kg. Therefore, we will give PKD mice nalfurafine daily at the maximum responsive dose 240 ug/kg/day for chronic studies in both Aim 1 and Aim 2 to obtain the greatest benefits.

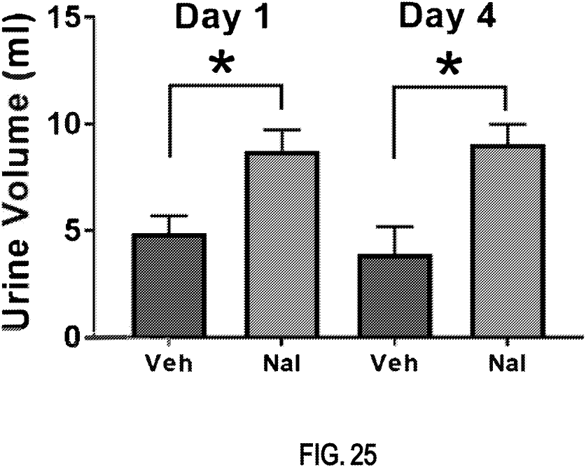

Route of administration: Drug will be given orally in drinking water. We have performed a study to test the stability of the drug. Fresh drug dissolved in water was given to a set of rats on the first day and it produced diuresis. We then left this water bottle at room temperature for three days, and we then gave this water to the same rats again. As shown in FIG. 25, the magnitude of increase in urine output is the same on day 4 as it was on day 1. Therefore, the drug is stable for 3 days when it is dissolved in water at room temperature. We will make fresh nalfurafine in drinking water every 3 days in the chronic studies in both Aim 1 and Aim 2. We will give another group of mice the standard care of tolvaptan to serve as comparison of the treatment. Due to poor water solubility of tolvaptan¹⁹, we will adopt the published route of administration of tolvaptan in Pkd1^{RC/RC} mice: mixing it in rodent chow at the concentration of 0.1%.²⁰

### Specific Aim 1: Determine the Effects of Nalfurafine on Reducing Cystogenesis During the Early Stage of PKD

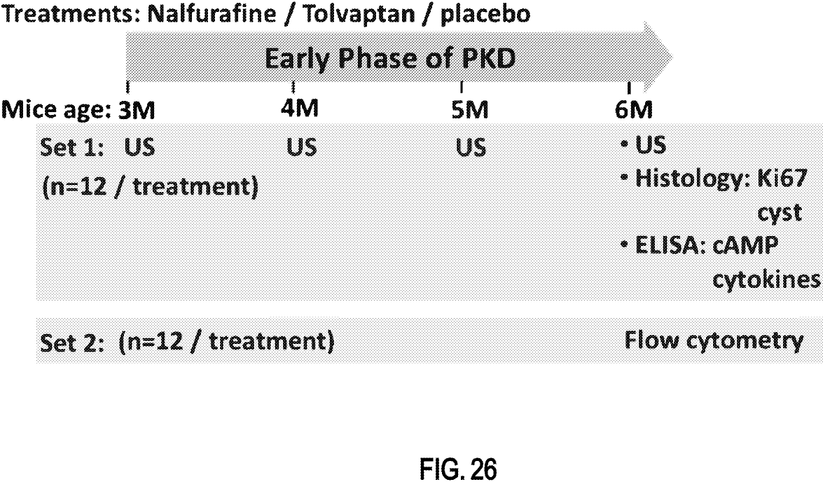

Hyperproliferation of epithelial cells in renal tubules is the primary pathology to cause formation of fluid-filled cysts. The formation and enlargement of cysts lead to an increase in the total kidney volume (KV) in the early phase of disease. ¹⁸ Infiltration of immune cells also accelerates the secretion of cyst fluid and proliferation of epithelial cells in the renal tubules. In the early phase of disease, the kidneys can maintain GFR at normal levels, and this will be studied in later stages of disease in Aim 2. The experimental design for Specific Aim 1 is outlined in FIG. 26.

Experiment 1a: Total kidney volume. We will have three groups of 3-month old male PKD mice (n=12 for each group). Sample size in each group is calculated using GPower software, and based on the urine output data and our experience with mice studies. Twenty-four-hour water intake will be measured for 3 consecutive days (Monday to Wednesday for every week), and it will be used to calculate the drug concentration in drinking water used in that week. One group will receive nalfurafine (240 ug/kg/day in water), the second group of mice will receive tolvaptan (0.1% diet), and the last control group of mice will receive diet containing placebo and regular drinking water with no drug added. Kidney volume will be measured using ultrasound (Vevo 3100, Visualsonics) when mice are 3, 4, 5, and 6 months old.

Experiment 1b: Inflammatory markers in the early phase of disease course. Macrophages accumulate in ADPKD kidneys and promote cyst growth. For these studies, three groups of mice (n=12/group) will be treated the same as outlined in Experiment 1a (Set 2 in FIG. 25). When these mice are 6 months old, we will sacrifice the mice and isolate total leukocytes in the kidneys to perform flow cytometry to determine macrophage numbers and subtypes. The markers used to identify monocytes, macrophages and its subtypes are: CD86, CD69, MHCII, CD163, CD11b, CD43.

Experiment 1c: Proliferation of tubular epithelial cells and evaluation of cysts. We will sacrifice the three groups of mice in Set 1 when they are 6 months old, and then perfuse the kidneys. One kidney will be fixed in zinc buffered formalin, embedded in paraffin and sectioned for histology staining. We will collaborate with LSUHSC Morphology and Imaging Core laboratory for this part of our studies. The other kidney will be flash frozen and used for molecular biology testing. Ki-67 is a cell cycle-associated protein and expressed in G1,S,G2 and M phase.²¹ It is a marker widely used to assess tubular epithelial cell proliferation on PKD animal models.²² We will perform immunohistochemical staining of Ki-67 to validate cell proliferation on kidney sections of mice treated with nalfurafine, tolvaptan or vehicle. Cysts numbers and size will be evaluated from H&E staining of the kidney sections. The slides will be evaluated by a pathologist blinded to the group, and cyst index will be used to compare the difference among groups of treatments.

Experiment 1d: Determine cAMP pathway and other inflammatory cytokines in the early phase of disease course. Monocyte chemoattractant protein-1 (MCP-1) is an important chemokines that attract monocytes to infiltrate into kidneys and later differentiate into macrophages to induce tubular injury in mice model of ADPKD.²³ MCP-1, TNFα, and cAMP levels will be measured using ELISA in the homogenate of the flash frozen kidney in Set 1 of mice.

### Outcomes

Without wishing to be bound by theory, nalfurafine will decrease kidney volume more than tolvaptan. Nalfurafine will lead to a significant reduction in the infiltrated monocytes and macrophages, which is correlated with a reduction in levels of proinflammatory cytokines and cell proliferation. In embodiments, we can combine kidneys from two mice together to have plenty of leukocytes to run flow cytometry. This can require treating another set of mice with n of 12 in the three groups.

### Specific Aim 2: Determine the Effects of Nalfurafine vs Tolvaptan on the Decline of Renal Function (GFR) and Fibrosis Severity in the Later Stages of PKD

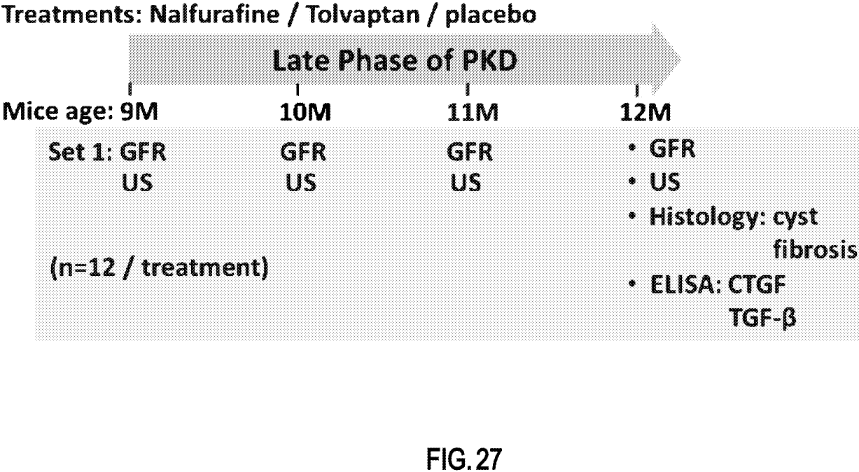

Although growing cysts continually replace normal nephrons, the remaining nephrons are still able to compensate for the loss and maintain normal GFR in the early phase of ADPKD. When the kidneys reach their limit of compensation, GFR will start to decline quickly and patients enter into ESRD much faster compared to the decades of the entire disease course.^{2,24,25} However, the data regarding GFR decline patterns in most mice models are lacking. This can be attributed to several reasons. First, in preclinical studies traditional GFR measurement is made by determining the renal clearance of inulin, which requires an invasive and non-survival procedure. Second, the litter size of PKD animals is small compared to normal animals. Third, Pkd1^{RC/RC} mice is a slow cysto-generous model and requires several months of growth to begin experiments. These facts lead to the missing information of a critical and highly clinically relevant parameter. We recently purchased innovative devices (MediBeacon) to measure GFR in conscious and free-moving mice with a non-invasive procedure, allowing multiple and time-course measurements of GFR on the same animal.²⁶ Fibrosis is another rapidly developing pathology in the late stage of PKD. Therefore, we will carry out studies to determine GFR and fibrosis in 9-12 months old Pkd1^{RC/RC} mice and validate whether nalfurafine delays the decline in GFR and reduces renal fibrosis. The experimental design for Aim 2 is outlined in FIG. 27.

Experiment 2a: Measure GFR. We will have 3 groups of 9-month-old male mice (n=12 each), and these mice will receive treatments as described in Experiment 1a. The GFR for each mouse will be measured at months 9, 10, 11, and 12. Mice will be anesthetized with isoflurane. The back of mice will be shaved and the transdermal mini GFR monitor will be taped on the back of mice. Mice will be injected with water soluble FITC-labeled sinistrin (5 mg/kg), which is only cleared by the kidneys. Mice will return to their home cage to recover from anesthesia. We will record real-time FITC fluorescence signal for 90 minutes on each mouse. This technique is proven to be as or more accurate than the traditional inulin measurement in preclinical studies, and it is currently under clinical trials for use to measure GFR in the USA. GFR will be measured on the same mice at months 9, 10, 11, and 12.

Experiment 2b: Total kidney volume. Ultrasound measurement of total kidney volume will be performed on mice when they are 9, 10, 11, and 12 months old in our Cardiovascular Core.

Experiment 2c: Fibrosis and cyst histology. We will sacrifice the mice at the end of the study, and kidneys will be perfused. One kidney will be fixed in zinc buffered formalin, paraffin embedded, and sectioned for staining. The other kidney will be flash frozen for molecular biology testing. Trichrome staining will be performed on these slides to evaluate the fibrosis. A pathologist blinded to treatment group will provide scoring of the fibrosis on each slide. In addition, the size and number of cysts will be evaluated with H&E staining by the same core laboratory. The slides will be evaluated by the pathologist blinded to the group, and cyst index will be used to compare the difference between groups of treatments.

Experiment 2d: ELISA for fibrosis markers. Connective tissue growth factor (CTGF) is a central mediator of fibrosis and tissue remodeling.²⁷ High levels of transforming growth factor β (TGF-β) and CTGF expression are highly corelated to fibrosis severity. We will use ELISA to measure CTGF and TGF-β levels in the homogenate of kidney tissue.

### Outcomes

Without wishing to be bound by theory, mice treated with nalfurafine will show a slower decline of GFR in the later phase of ADPKD compared to mice treated with tolvaptan or vehicle. Nalfurafine will decrease the fibrosis severity and pro-fibrotic marker levels in the kidneys. In embodiments, kidney tissue can be replaced by fibrosis and the production of CTGF and TGF-β can slow down significantly. Thus, embodiments can comprise treating another set of mice and sacrificing the mice at 10 months old to catch the much higher expressive phase of CTGF and TGF-β, and to validate whether nalfurafine treatment will decrease these fibrotic markers in later phases of ADPKD disease course.

### Statistical Analysis

Results will be expressed as mean ± SEM. Two-way ANOVA will be used to compare GFR and ultrasound data among three treatment groups (nalfurafine vs tolvaptan vs placebo) with the two factors of treatment and time. One-way ANOVA will be used to compare ELISA and flowcytometry data among treatment groups. Wilcoxon signed-rank test will be used to compare the histology data among groups of treatments. Statistical analysis will be performed using GraphPad Prism software. Justification for the number of animals is provided in the Vertebrate Animals section.

We are using only male mice in this study for two reasons: 1) we need to keep the females as breeders; 2) minimize the variance. Gender difference can also be validated.

### Scientific Rigor and Reproducibility

We will collect data from multiple mice (n>10) for each experiment. Appropriate sample sizes will be determined using power analysis. Experimental parameters will be carefully controlled and clearly described. Data collected will be included in the final analysis. Critical findings will be replicated in randomized double-blinded experiments.

### References Cited in This Example

1. NIDDK. https://www.niddk.nih.gov/about-niddk/advisory-coordinating-committees/kuhicc/kicc/federal-ckd-matrix/veterans-affairs-va-activities.

2. Horie S, Mochizuki T, Muto S, Hanaoka K, Fukushima Y, Narita I, Nutahara K, Tsuchiya K, Tsuruya K, Kamura K, Nishio S, Suwabe T, Ubara Y, Ishimura E, Nakanishi K, Furukawa K, Kimura K, Matsuo S. Evidence-based clinical practice guidelines for polycystic kidney disease 2014. Clin Exp Nephrol. 2016; 20:493-509. <https://doi.org/10.1007/sl0157-015-1219-7>.

3. Blair HA. Tolvaptan: A Review in Autosomal Dominant Polycystic Kidney Disease. Drugs. 2019; 79:303-313. <https://doi.org/10.1007/s40265-019-1056-1>.

4. Kumagai H, Ebata T, Takamori K, Miyasato K, Muramatsu T, Nakamoto H, Kurihara M, Yanagita T, Suzuki H. Efficacy and safety of a novel κ-agonist for managing intractable pruritus in dialysis patients. Am J Nephrol. 2012; 36:175-183. <https://doi.org/10.1159/000341268>.

5. Kozono H, Yoshitani H, Nakano R. Post-marketing surveillance study of the safety and efficacy of nalfurafine hydrochloride (Remitch® capsules 2.5 µg) in 3,762 hemodialysis patients with intractable pruritus. Int J Nephrol Renovasc Dis. 2018; 11:9-24. <https://doi.org/10.2147/IJNRD.S145720>.

6. Menon V, Rudym D, Chandra P, Miskulin D, Perrone R, Sarnak M. Inflammation, oxidative stress, and insulin resistance in polycystic kidney disease. Clin J Am Soc Nephrol. 2011; 6:7-13. <https://doi.org/10.2215/CJN.04140510>.

7. Chebib FT, Sussman CR, Wang X, Harris PC, Torres VE. Vasopressin and disruption of calcium signalling in polycystic kidney disease. Nat Rev Nephrol. 2015; 11:451-464. <https://doi.org/10.1038/nmeph.2015.39>.

8. Chen L, Zhou X, Fan LX, Yao Y, Swenson-Fields KI, Gadjeva M, Wallace DP, Peters DJM, Yu A, Grantham JJ, Li X. Macrophage migration inhibitory factor promotes cyst growth in polycystic kidney disease. J Clin Invest. 2015; 125:2399-2412. <https://doi.org/10.1172/JCI80467>.

9. Eisenstein TK. The Role of Opioid Receptors in Immune System Function. Front Immunol. 2019; 10:2904. <https://doi.org/10.3389/fimmu.2019.02904>.

10. Zhang S, Zhou Y, Zhao L, Tian X, Jia M, Gu X, Feng N, An R, Yang L, Zheng G, Li J, Guo H, Fan R, Pei J. κ-opioid receptor activation protects against myocardial ischemia-reperfusion injury via AMPK/Akt/eNOS signaling activation. Eur J Pharmacol. 2018; 833:100-108. <https://doi.org/10.1016/j.ejphar.2018.05.043>.

11. Elliott G, Vanwersch R, Soeberdt M, Metze D, Lotts T, Ständer S, Abels C. Topical nalfurafine exhibits anti-inflammatory and anti-pruritic effects in a murine model of AD. J Dermatol Sci. 2016; 84:351-354. <https://doi.org/10.1016/j.jdermsci.2016.09.008>.

12. Torres VE, Harris PC. Strategies targeting cAMP signaling in the treatment of polycystic kidney disease. J Am Soc Nephrol. 2014; 25:18-32. <https://doi.org/10.1681/ASN.2013040398>.

13. Yamaguchi T, Nagao S, Kasahara M, Takahashi H, Grantham JJ. Renal accumulation and excretion of cyclic adenosine monophosphate in a murine model of slowly progressive polycystic kidney disease. Am J kidney Dis Off J Natl Kidney Found. 1997; 30:703-709. <https://doi.org/10.1016/s0272-6386(97)90496-0>.

14. Kip SN, Hunter LW, Ren Q, Harris PC, Somlo S, Torres VE, Sieck GC, Qian Q. [Ca2+]i reduction increases cellular proliferation and apoptosis in vascular smooth muscle cells: relevance to the ADPKD phenotype. Circ Res. 2005; 96:873-880. <https://doi.org/10.1161/01.RES.0000163278.68142.8a>.

15. Liu L-J, Yu J-J, Xu X-L. Kappa-opioid receptor agonist U50448H protects against renal ischemiareperfusion injury in rats via activating the PI3K/Akt signaling pathway. Acta Pharmacol Sin. 2018; 39:97-106. <https://doi.org/10.1038/aps.2017.51>.

16. Zhou X, Wang D, Zhang Y, Zhang J, Xiang D, Wang H. Activation of κ-opioid receptor by U50,488H improves vascular dysfunction in streptozotocin-induced diabetic rats. BMC Endocr Disord. 2015; 15:7. <https://doi.org/10.1186/sl2902-015-0004-7>.

17. Rossi NF, Brooks DP. kappa-Opioid agonist inhibition of osmotically induced AVP release: preferential action at hypothalamic sites. Am J Physiol. 1996; 270:E367-72. <https://doi.org/10.1152/ajpendo.1996.270.2.E367>.

18. Hopp K, Ward CJ, Hommerding CJ, Nasr SH, Tuan HF, Gainullin VG, Rossetti S, Torres VE, Harris PC. Functional polycystin-1 dosage governs autosomal dominant polycystic kidney disease severity. J Clin Invest. 2012; 122:4257-4273. <https://doi.org/10.1172/JCI64313>.

19. National Center for Biotechnology Information. Tolvaptan, CID=216237, https://pubchem.ncbi.nlm.nih.gov/compound/Tolvaptan.

20. Hopp K, Hommerding CJ, Wang X, Ye H, Harris PC, Torres VE. Tolvaptan plus pasireotide shows enhanced efficacy in a PKD1 model. J Am Soc Nephrol. 2015; 26:39-47. <https://doi.org/10.1681/ASN.2013121312>.

21. Romar GA, Kupper TS, Divito SJ. Research techniques made simple: Techniques to assess cell proliferation. J Invest Dermatol. 2016; 136:el-e7. <https://doi.org/10.1016/j.jid.2015.11.020>.

22. Wallace DP, White C, Savinkova L, Nivens E, Reif GA, Pinto CS, Raman A, Parnell SC, Conway SJ, Fields TA. Periostin promote renal cyst growth and interstital fibrosis in polycystic kidney disease. Kidney Int. 2014; 85:845-854. https://doi.org/10.1016/j.physbeh.2017.03.040.

23. Zheng D, Wolfe M, Cowley BDJ, Wallace DP, Yamaguchi T, Grantham JJ. Urinary excretion of monocyte chemoattractant protein-1 in autosomal dominant polycystic kidney disease. J Am Soc Nephrol. 2003; 14:2588-2595. <https://doi.org/10.1097/01.asn.0000088720.61783.19>.

24. Tan AY, Blumenfeld J, Rennert H. Polycystic kidney disease. Mol Pathol Clin Pract Ed. 2016:277-290. <https://doi.org/10.1007/978-3-319-19674-9_19>.

25. Brosnahan GM, Abebe KZ, Moore CG, Rahbari-Oskoui FF, Bae KT, Grantham JJ, Schrier RW, Braun WE, Chapman AB, Flessner MF, Harris PC, Hogan MC, Perrone RD, Miskulin DC, Steinman TI, Torres VE. Patterns of Kidney Function Decline in Autosomal Dominant Polycystic Kidney Disease: A Post Hoc Analysis From the HALT-PKD Trials. Am J kidney Dis Off J Natl Kidney Found. 2018; 71:666-676. <https://doi.org/10.1053/j.ajkd.2017.10.023>.

26. Lakshmipathi J, Gao Y, Hu C, Stuart D, Genzen J, Ramkumar N, Kohan DE. Nephron-Specific Disruption of Polycystin-1 Induces Cyclooxygenase-2-Mediated Blood Pressure Reduction Independent of Cystogenesis. J Am Soc Nephrol. April 2020. <https://doi.org/10.1681/ASN.2019090934>.

27. Lipson KE, Wong C, Teng Y, Spong S. CTGF is a central mediator of tissue remodeling and fibrosis and its inhibition can reverse the process of fibrosis. Fibrogenesis Tissue Repair. 2012; 5:2-9. <https://doi.org/10.1186/1755-1536-5-sl-s24>.

### EQUIVALENTS

Those skilled in the art will recognize, or be able to ascertain, using no more than routine experimentation, numerous equivalents to the specific substances and procedures described herein. Such equivalents are considered to be within the scope of this invention, and are covered by the following claims.

What is claimed:

1. A method of treating a subject afflicted with polycystic kidney disease (PKD), the method comprising administering to a subject a therapeutically effective amount of a kappa opioid receptor agonist (KOA).

2. The method of claim 1, wherein the kappa opioid receptor agonist decreases circulating levels of vasopressin.

3. The method of claim 1, where the kappa opioid receptor agonist comprises an organic small molecule.

4. The method of claim 1, where a therapeutically effective amount of a kappa opioid receptor agonist (KOA) is between 0.005 mg/kg to 10 mg/kg.

5. The method of claim 2, where the organic small molecule is JT09, nalfurafine, CR665, or CR845.

6. The method of claim 2, where the organic small molecule comprises a molecule of:. 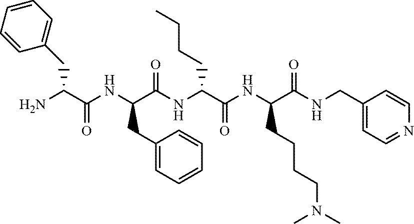 [SMILES: CCCC[C@@H](NC(=O)[C@@H](Cc1ccccc1)NC(=O)[C@H](N)Cc1ccccc1)C(=O)N[C@H](CCCCN(C)C)C(=O)NCc1ccncc1]
.

7. The method of claim 2, where the organic small molecule comprises a molecule of: 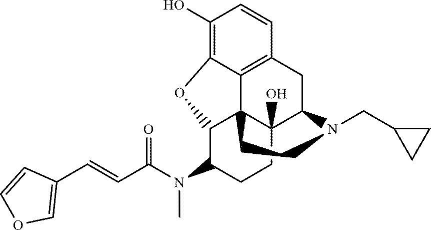
.

8. The method of claim 2, where the organic molecule comprises a molecule of: 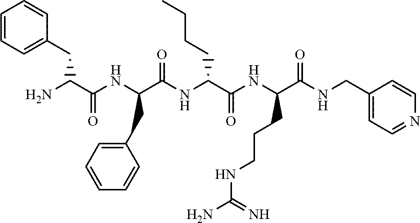
.

9. The method of claim 2, where the organic molecule comprises a molecule of:. 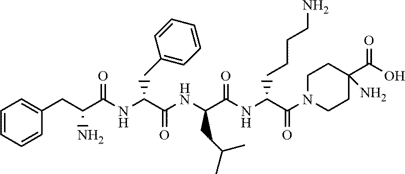 [SMILES: CC(C)C[C@@H](NC(=O)[C@@H](Cc1ccccc1)NC(=O)[C@H](N)Cc1ccccc1)C(=O)N[C@H](CCCCN)C(=O)N1CCC(N)(C(=O)O)CC1]
.

10. The method of claim 1, where the kappa opioid receptor agonist is administered orally or intravenously.

11. The method of claim 1, where the kappa opioid receptor agonist is administered by bolus injection, or by infusion, or by inhalation, or by intranasal administration.

12. The polycystic kidney disease of claim 1, wherein the PKD comprises autosomal dominant polycystic kidney disease (ADPKD) or autosomal recessive polycystic kidney disease.

13. The method of claim 1, wherein treating comprises decreasing circulating blood levels of vasopressin, maintaining glomerular filtration rate, reducing kidney fibrosis, decreasing blood urea levels, decreasing cysts number, decreasing cysts size, decreasing serum creatinine levels, or a combination thereof.

14. The method of claim 1, wherein the kappa opioid receptor agonist is administered chronically.

15. The method of claim 1, wherein the kappa opioid receptor agonist (KOA) is administered as a pharmaceutical composition.
